SEQUENCE LISTING (17 sequences)

SEQ ID NO: 1 - protein, 201 aa, Homo sapiens
MAWMLLLILIMVHPGSCALWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTVLLLRAGFYAVSFLSVAVGSTVYYQGKCLTWKGPRRQLPAVVPAPLPPPCGSSAHLLPPVPGG

SEQ ID NO: 2 - protein, 276 aa, Homo sapiens
MAWRALHPLLLLLLLFPGSQAQSKAQVLQSVAGQTLTVRCQYPPTGSLYEKKGWCKEASALVCIRLVTSSKPRTMAWTSRFTIWDDPDAGFFTVTMTDLREEDSGHYWCRIYRPSDNSVSKSVRFYLVVSPASASTQTPWTPRDLVSSQTQTQSCVPPTAGARQAPESPSTIPVPSQPQNSTLRPGPAAPIALVPVFCGLLVAKSLVLSALLVWWGDIWWKTVMELRSLDTQKATCHLQQVTDLPWTSVSSPVEREILYHTVARTKISDDDDEHTL

SEQ ID NO: 3 - protein, 304 aa, Homo sapiens
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRMGLAFLVLVALVWFLVEDWLSRKRTRERASRASTWEGRRRLNTQTL

SEQ ID NO: 4 - protein, 366 aa, Artificial
  Fusion protein
WVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTVLLLRAGFYAVADPEEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 366 aa, Artificial
  Fusion protein
WVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTVLLLRAGFYAVADPEGERTYRVPRDCGCKPCICVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 6 - protein, 367 aa, Artificial
  Fusion protein
LWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTVLLLRAGFYAVADPEGERTYRVPRDCGCKPCICVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 7 - protein, 361 aa, Artificial
  Fusion protein
WVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTIEGRWMQDPEGERTYRVPRDCGCKPCICVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 8 - protein, 362 aa, Artificial
  Fusion protein
LWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTIEGRWMQDPEGERTYRVPRDCGCKPCICVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 9 - protein, 347 aa, Artificial
  Fusion protein
ALWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTLEHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 350 aa, Artificial
  Fusion protein
ALWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTLEVPRDCGCKPCICTVPEVSSVFIFPPKPKDVLTITLTPKVTCVVVDISKDDPEVQFSWFVDDVEVHTAQTKPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 11 - protein, 126 aa, Artificial
  Fusion protein
LWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTIEGRMD

SEQ ID NO: 12 - protein, 351 aa, Artificial
  Fusion protein
LWVSQPLEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGDPEPKSSDKTHTCPPCPAPEFEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 231 aa, Homo sapiens
MQDEERYMTLNVQSKKRSSAQTSQLTFKDYSVTLHWYKILLGISGTVNGILTLTLISLILLVSQGVLLKCQKGSCSNATQYEDTGDLKVNNGTRRNISNKDLCASRSADQTVLCQSEWLKYQGKCYWFSNEMKSWSDSYVYCLERKSHLLIIHDQLEMAFIQKNLRQLNYVWIGLNFTSLKMTWTWVDGSPIDSKIFFIKGPAKENSCAAIKESKIFSETCSSVFKWICQY

SEQ ID NO: 14 - protein, 205 aa, Homo sapiens
MSRGLQLLLLSCAYSLAPATPEVKVACSEDVDLPCTAPWDPQVPYTVSWVKLLEGGEERMETPQEDHLRGQHYHQKGQNGSFDAPNERPYSLKIRNTTSCNSGTYRCTLQDPDGQRNLSGKVILRVTGCPAQRKEETFKKYRAEIVLLLALVIFYLTLIIFTCKFARLQSIFPDFSKAGMERAFLPVTSPNKHLGLVTPHKTELV

SEQ ID NO: 15 - protein, 199 aa, Homo sapiens
MSSENCFVAENSSLHPESGQENDATSPHFSTRHEGSFQVPVLCAVMNVVFITILIIALIALSVGQYNCPGQYTFSMPSDSHVSSCSEDWVGYQRKCYFISTVKRSWTSAQNACSEHGATLAVIDSEKDMNFLKRYAGREEHWVGLKKEPGHPWKWSNGKEFNNWFNVTGSDKCVFLKNTEVSSMECEKNLYWICNKPYK

SEQ ID NO: 16 - DNA, 42 bp, Artificial
  Primer
cactgcagctagcactctgggtgtcccagccccctgagattc

SEQ ID NO: 17 - DNA, 52 bp, Artificial
  Primer
ccagcaagatctgcatccatcggccttcgattgtaccagcccctagctgagg

(19) United States
(12) Patent Application Publication
(10) Pub. No.: US 2009/0010843 A1
(43) Pub. Date: Jan. 8, 2009

(54) Methods of Identifying Antibodies to Ligands of Orphan Receptors

(75) Inventors: Pieter Spee, Allerod (DK); Peter Andreas Wagtmann, Rungsted Kyst (DK); Thomas Chin Che Tan, Copenhagen (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 12/067,686

(22) PCT Filed: Sep. 25, 2006
(86) PCT No.: PCT/EP2006/066697

§ 371 Date: Jun. 25, 2008

Related U.S. Application Data
(60) Provisional application No. 60/721,014, filed on Sep. 27, 2005.
(60) Provisional application No. 60/721,014, filed on Sep. 27, 2005.
(60) Provisional application No. 60/721,014, filed on Sep. 27, 2005.
(60) Provisional application No. 60/721,014, filed on Sep. 27, 2005.

(30) Foreign Application Priority Data
Sep. 23, 2005 (EP) 05108816.9

(51) Int. Cl. A61K 51/10; G01N 33/53; C12P 21/04; G01N 33/00; C07K 16/00; C12N 5/02; A61K 39/395

(52) U.S. Cl. 424/1.49; 435/7.2; 435/71.1; 435/69.6; 435/7.24; 530/387.3; 435/375; 424/134.1; 424/178.1

(57) ABSTRACT

Described is a method of identifying antibodies against hitherto unknown ligands of orphan receptors or other orphan ligands, i.e., receptors or other ligands where the counter-ligand has not yet been identified. The availability of antibodies binding to the unknown ligand significantly facilitates their isolation and characterization, and the identified antibodies can themselves be useful for treating patients with cancer or autoimmune diseases, or other disorders. An exemplary embodiment provides for a method designated Identification of Therapeutic Antibodies by Competitive Screening (ITACS). Described are also fusion proteins comprising a soluble portion of an orphan receptor, such as NKp30, and the Fc portion of an antibody. The fusion proteins typically comprise a Flexible Transmembrane Linker (FTL), i.e., a linker comprising a portion of a transmembrane domain of the orphan receptor.

### FIELD OF THE INVENTION

The present invention relates to the identification of cell surface-associated ligands to orphan ligands such as, e.g., orphan receptors, and antibodies or other agents against such cell surface-associated ligands, as well as to their use in methods treating various conditions and diseases.

### BACKGROUND OF THE INVENTION

Natural killer (NK) cells play a dominant role in immune-surveillance of tumors and viral infections. It was long believed that NK cells were activated by default via activating receptors when encountering target cells, and that the choice of whether to kill or spare a potential target cell was controlled by NK-cell inhibitory receptors. Recent studies, however, suggest that NK cells kill only sick or abnormal cells, but not healthy ones, even in the absence of inhibitory signaling. This suggests that ligands for activating NK receptors may be predominantly expressed by abnormal, sick, stressed, or infected target cells. For example, expression of MICA and MICB, which are ligands for the activating NK cell receptor NKG2D, are absent from most normal tissues, but can be induced by viral and bacterial infections and are expressed by many tumors of epithelial origin.

Natural Killer cell p30 related protein, also known as NKp30, BMOG, 1C7, HGNC:14189, LY117, and natural cytotoxicity triggering receptor 3, is one of the main NK-cell activating receptors, functioning as an important factor in determining the NK-mediated killing of target cells. Other major receptors responsible for NK cell triggering include NKp44 and NKp46 (Moretta and Moretta (EMBO J 2004; 23:255-9)). It has been shown that NKp30, NKp44, and NKp46 are involved in NK cell-mediated killing of several types of tumor cells, such as, e.g., leukemias and lymphomas, melanomas, lung adenocarcinomas, neuro- and glioblastomas, and/or hepatocarcinomas, and that, in many cases, such killing can be inhibited or reduced by antibodies against one or more of these receptors (for example, see Castriconi et al, Cancer Res. 2004; 64(24):9180-4.; Pende et al., Blood. 2005 105(5):2066-73, Pende et al, J Exp Med. 1999 Nov. 15; 190(10):1505-16. Reviewed in Moretta et al., Annual Review of Immunology Vol. 19: 197-223).

The respective ligands for NKp30, NKp44, and NKp46 (herein denoted NKp30L, NKp44L, and NKp46L, respectively) could represent alternative and useful therapeutic targets for treatment of cancer and other disorders where activation of these receptors plays a role. Viral proteins, such as hemagglutinin, have been implicated in serving as ligands for NKp44 and NKp46 (Mandelboim et al, Nature, Vol. 409 (6823) pp. 1055-1060 (2001), Amon et al. European journal of immunology, Vol. 31 (9) pp. 2680-2689 (2001)), but no naturally expressed NKp30L, NKp44L, and NKp46L, including e.g. stress- or cancer-associated NKp30L, NKp44L, and NKp46L, have been identified to date. Whereas functional screening and biological assays relying on the ability of agents to diminish NK cell activation have identified antibodies to NKp30, NKp44, NKp46, and to a purported NKp44-ligand related to virus infection (Vieillard), such assays are usually not amenable to high-throughput screening. Fusion proteins between the NKp30, NKp44, or NKp46 receptors and immunoglobulin Fc domains (see, e.g., WO9923867, WO200208287, WO2004053054, WO2005000086, WO2005051973, and R&D Systems Inc., Catalog No. 1849-NK) can bind the NKp30L, NKp44L, and NKp46L, respectively. Preparing such fusion proteins can be a challenge, however, since soluble receptors often bind to cell-surface ligands with relatively low affinity, limiting their usefulness for therapeutic applications.

Therapies directed against the cell surface-associated ligands for activating NK cell receptors and other orphan ligands have thus so far been hampered by the fact that the identities of many such cell surface-associated ligands are still unidentified. For example, monoclonal antibodies binding the ligands of orphan receptors are difficult to identify, since traditional antibody production typically relies on immunization of an experimental animal with a known and characterized antigen. In the absence of the antigen itself, alternative methods can be based on, for example, in vitro immunization of human B-cells, using immobilized cells as antigen (see, e.g., U.S. Pat. No. 6,541,225 and EP0218158). Such methods, however, require access to large amounts of human B-cell populations and yield antibodies against a range of cellular antigens.

Accordingly, there is a need in the art for convenient methods to identify cell-associated ligands to orphan NK cell-receptors and other orphan members of ligand pairs, as well as antibodies and other targeting agents against such cell-associated ligands. The present invention addresses these and other needs in the art.

### SUMMARY OF THE INVENTION

The present invention provides methods of producing and identifying antibodies against antigens that can be hitherto unknown ligands of orphan receptors or other orphan ligands, i.e., receptors or other ligands where the counter-ligand has not yet been identified. In one aspect, such a method comprises immunizing an animal with a preparation of target cells (e.g., cells to which an orphan ligand binds), and identifying any antibodies generated by the animal which compete with the orphan ligand in binding to target cells. An exemplary embodiment of such a method, designated Identification of Therapeutic Antibodies by Competitive Screening (ITACS), is depicted in FIG. 1.

The present invention also provides fusion or hybrid proteins comprising a soluble portion of a receptor such as, e.g., NKp30, and an Fc portion of an IgG antibody. In one aspect, the fusion or hybrid proteins comprise a portion of a Flexible Transmembrane Linker (FTL), i.e., a linker comprising a portion of a transmembrane domain of the orphan receptor.

These and other aspects, features, and embodiments of the invention are described in further detail herein.

### BRIEF DESCRIPTION OF THE DRAWINGS

FIG. 1 depicts an overview of an exemplary Identification of Therapeutic Antibodies by Competitive Screening (ITACS) procedure.

FIG. 2 depicts a novel soluble NKp30-Fc fusion protein comprising a flexible transmembrane-derived linker (FTL) linked to the Fc domain of human IgG1 (SEQ ID NO:4). The fusion protein is hereafter generally referred to as soINKp30-FTL-Fc, or specifically referred to as soINKp30-FTL-hFc (SEQ ID NO:4) or soINKp30-FTL-mFc (SEQ ID NO:5) to indicate fusion proteins where the Fc portion is of human (h) or murine (m) origin. The shaded sequence indicates the NKp30-portion, also including an additional alanine (A) residue.

FIG. 3 depicts FACS data on the binding of soINKp30-FTL-Fc to K562 cells. (A) Background binding of the secondary APC-conjugated donkey anti-human Fc Ab to K562 cells in the absence soINKp30-FTL-hFc. (B) Binding of soINKp30-FTL-hFc (15 ug/ml) to K562 cells, detected by the secondary APC-conjugated donkey anti-human Fc.

FIG. 4 depicts a FACS comparison between soINKp30-FTL-Fc and a commercially available soINKp30-Fc protein from R&D Systems, Inc. (Catalog No. 1849-NK) in binding to K562 cells, showing improved intensity of staining, reflecting increased strength of binding, of soINKp30-FTL-Fc to K562 cells. (A) Binding of soINKp30-FTL-hFc to K562 cells. (B) Binding of 1849-NK to K562 cells. In both A and B, identical amounts of soluble NKp30 proteins were added to the cells, and the binding was revealed by the secondary APC-conjugated donkey anti-human Fc.

FIG. 5 depicts a competitive FACS study between soINKp30-FTL-Fc and the anti-human NKp30 mAb cl45 (R & D systems), showing that cl45 inhibits soINKp30-FTL-Fc from binding to K562 cells. (A) Staining by soINKp30-FTL-hFc (15 ug/ml) detected by the secondary APC-conjugated donkey anti-human Fc; (B) Staining by soINKp30-FTL-hFc in the presence of 45 μg/ml of cl45. (C) Staining by soINKp30-FTL-hFc in the presence of 90 μg/ml cl45. (D) Staining by soINKp30-FTL-hFc in the presence of 180 μg/ml cl45. The binding of soINKp30-FTL-hFc was not competed by irrelevant control mAbs.

FIG. 6 depicts an amino acid sequence alignment of soINKp30-FTL-hFc (top, SEQ ID NO:4) with the NKp30 portion of the commercially available soINKp30-Fc construct 1849-NK8 (middle, SEQ ID NO:11) and a soINKp30-Fc construct described in WO 2004/053054 (bottom, SEQ ID NO:12). The “W” residue of the soINKp30-FTL-hFc fusion protein (residue 2 according to the amino acid numbering in the figure) corresponds to residue No. 20 in full-length NKp30 (SEQ ID NO:1).

FIG. 7 shows the comparative binding of soINKp30-FTL-mFc and NKp30-mFc(c) to K562 cells. Filled histogram: IgG1 control mAb, dotted line: soINKp30-mFc(c), solid line: soINKp30-FTL-mFc; all at 20 μg/ml.

FIG. 8 shows the result of an ITACS-screen for mAbs to NKp30L, identifying an anti-NKp30L mAb. K562 cells were incubated with or without supernatants from hybridomas made from a mouse immunized with K562, followed by soINKp30-FTL-hFc. Binding of the latter was detected using a secondary APC-conjugated donkey anti-human Fc Ab. (A) Binding of soINKp30-FTL-hFc to K562 cells in the absence of hybridoma supernatant, detected by APC-conjugated donkey anti-hFc. (B) Binding of soINKp30-FTL-hFc to K562 cells in the presence of a hybridoma supernatant which was designated to be a negative clone, since the binding of soINKp30-FTL-hFc was not reduced by the hybridoma supernatant, as compared to the staining in panel A. (C) Binding of soINKp30-FTL-hFc to K562 cells in the presence of a hybridoma supernatant which was designated to be a positive clone, since this hybridoma supernatant reduced binding of the soINKp30-FTL-hFc protein.

### DEFINITIONS

A “ligand pair” is an entity comprising at least two ligands which detectably and selectively can bind each other. Numerous methods are known in the art for the detection of binding of a ligand pair, often based on one ligand being attached to a solid surface, cell, or bead, and one or more members of the ligand pair being labeled with a detectable moiety. Exemplary and non-limiting examples of ligand pairs include protein-protein, receptor-ligand, receptor-hormone, and antibody-antigen ligand pairs.

An “orphan ligand” of a ligand pair is a known member of a ligand pair where at least one other ligand is unidentified. One exemplary and non-limiting type of orphan ligand described herein is orphan receptors.

A “target ligand” is an unidentified ligand binding to an orphan receptor.

As used herein, a “receptor” is a cell-associated member of a ligand pair, where binding of the ligand to the receptor can result in one or more detectable effects on the cell. For the avoidance of doubt, a cell-surface bound ligand may also function as a receptor. Thus, for a particular ligand pair, both, one, or no members may be receptors. A “soluble receptor” is a portion of the receptor which can exist in solution, and which often comprises at least an extracellular portion of the receptor. Exemplary receptors described herein include NK cell activating and inhibitory receptors.

A “cell surface-associated ligand” is a cell-surface associated member of a ligand pair, where binding of the ligand pair can take place extracellularly.

As used herein, the term “antibody” means an antigen-binding protein comprising at least the antigen-binding portions of a monoclonal or polyclonal antibody, and includes, but is not limited to, full-length antibodies of the IgA, IgD, IgE, IgG (including IgG1, IgG2, IgG3, and IgG4 isotypes), and IgM type, as well as antibody fragments known in the art, including, e.g., Fab, F(ab)₂, F(ab′)₂, Fd, scFv, and dsFv fragments. The antibody can be of any origin, including, but not limited to, murine and human, and may be a modified version of a parent antibody or antibodies, including but not limited to, a chimeric, humanized, or single-chain antibody. Unless contradicted by context, the terms “antibody” and IgG” are used interchangeably herein.

As used herein, an “antibody fragment” comprises a portion of a full-length antibody, and is capable of binding an antigen. Typically, an antibody fragment comprises at least the CDR-region of an antibody. Exemplary antibody fragments include, but are not limited to, Fab, F(ab)₂, F(ab′)₂, Fd, scFv, and dsFv fragments.

As used herein, an “antibody derivative” is an antibody or antibody fragment conjugated or otherwise associated with a non-antibody peptide or a chemical compound that is not normally part of an antibody. Exemplary antibody derivatives are antibodies or antibody fragments conjugated to cytotoxic drugs or radionuclides.

An antibody that “blocks” the binding between a cell-surface-associated ligand of an orphan receptor is an antibody that reduces the binding of a soluble receptor or ligand (or, e.g., a soluble fragment or Fc construct thereof) by at least about 20%, at least about 30%, at least about 40% or at least about 50%, typically in a dose-dependent fashion. An exemplary assay for determining whether an antibody is capable of such blocking is provided in Example 4.

Terms such as “peptide,” “protein,” and “polypeptide” are to be understood to provide support for one another herein and to be amenable to interchangeable use generally, unless otherwise stated or contradicted by context. Furthermore, terms like “peptide” and “protein” used herein should generally be understood as referring to any suitable peptide of any suitable size and composition (e.g., with respect to the number of amino acids, number of associated chains in a protein molecule, overall size, etc.). Moreover, peptides in the context of the inventive methods and compositions described herein can comprise non-naturally occurring and/or non-L amino acid residues, unless otherwise stated or contradicted by context.

A “hybrid” protein is a protein comprising two polypeptide segments linked via at least one linkage other than a peptide bond (e.g., by chemical coupling or an affinity interaction such as via, e.g., biotin/avidin).

A “fusion” protein is a protein comprising two polypeptide segments linked by a peptide bond, produced, e.g., by recombinant processes.

In the context of the present invention, “treatment” or “treating” refers to preventing, alleviating, managing, curing or reducing one or more symptoms or clinically relevant manifestations of a disease or disorder, unless contradicted by context. For example, “treatment” of a patient in whom no symptoms or clinically relevant manifestations of a disease or disorder have been identified is preventive therapy, whereas “treatment” of a patient in whom symptoms or clinically relevant manifestations of a disease or disorder have been identified generally does not constitute preventive therapy.

A “therapeutically effective amount” refers to an amount effective, when delivered in appropriate dosages and for appropriate periods of time, to achieve a desired therapeutic result in a host For example, with respect to cancer treatment, a therapeutically effective amount can be an amount capable of reducing one or more aspects of cancer progression, increasing the likelihood of survival over a period of time (e.g., 18-60 months after initial cancer treatment), reducing the spread of cancer cell-associated growths, and/or reducing the likelihood of recurrence of tumor growth. A therapeutically effective amount can vary according to factors such as the disease state, age, sex, and weight of the individual, and the ability of a therapeutic agent to elicit a desired response in the individual. A therapeutically effective amount is also one in which any toxic or detrimental effects of the therapeutic agent portion are outweighed by the therapeutically beneficial effects.

A “prophylactically effective amount” refers to an amount effective, at dosages and for periods of time necessary, to achieve a desired prophylactic result (e.g., a reduction in the likelihood of developing a disorder, a reduction in the intensity or spread of a disorder, an increase in the likelihood of survival during an imminent disorder, a delay in the onset of a disease condition, a decrease in the spread of an imminent condition as compared to in similar patients not receiving the prophylactic regimen, etc.). Typically, because a prophylactic dose is used in subjects prior to or at an earlier stage of disease, the prophylactically effective amount will be less than the therapeutically effective amount.

Where the phrase “effective amount” is used without a modifier such as “therapeutically” or “prophylactically”, the phrase is intended to mean an amount that is at least as great as the minimum prophylactically effective or therapeutically effective amount and that is appropriate for the indicated use. The phrase “effective amount” encompasses both “prophylactically effective” and “therapeutically effective” amounts unless otherwise stated or clearly contradicted by context.

### DESCRIPTION OF THE INVENTION

This invention is based, in part, on the discoveries of a convenient and efficient method to produce and identify antibodies against unidentified cell-surface-associated “target” ligands for orphan NK cell receptors and other orphan ligands, and of constructs between a soluble ligand, e.g., a receptor, and an IgG Fc-domain, having superior properties over known constructs. The method is herein denoted Identification of Therapeutic Antibodies by Competitive Screening (ITACS), and is particularly applicable to identifying antibodies against hitherto unknown ligands to orphan receptors. However, in an alternative aspect, the same method steps are used to identify antibodies against known cell-surface associated ligands (i.e., in cases where the receptor is not orphan).

In one aspect, the ITACS method comprises the steps of:

(i) identifying a cell-line that expresses the unknown target ligand on its cell-surface, e.g., by testing for a cell-line to which the orphan receptor binds (“target cell”);

(ii) using a cell line identified in (i) (or a membrane-preparation thereof) to immunize a vertebrate animal, usually an experimental animal such as, e.g., a mouse or rat;

(iii) preparing antibody-producing cells from the vertebrate animal such as, e.g., hybridomas; and

(iv) screening for antibodies from the antibody-producing cells which compete with the orphan receptor in binding to a cell-line identified in (i).

The identified antibodies are characterized by their abilities to bind to the target ligand, and to block the interaction between the target ligand and the orphan receptor.

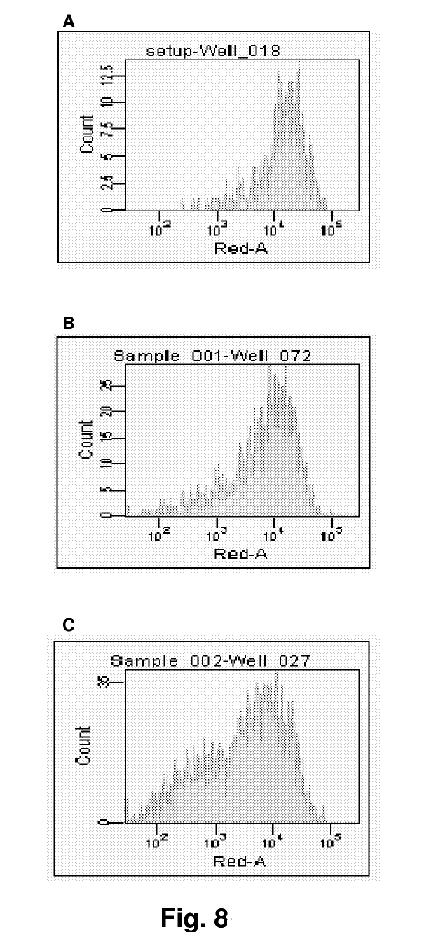

The method can advantageously be applied in a high-throughput format using, e.g., an Fmat scanner (PE Biosystems, CA), or a FACSarray (Beckton Dickinson, Calif.), or similar types of analyzers from other manufacturers. While sometimes described in the context of identifying antibodies to cell-associated ligands for orphan receptors or other orphan ligands, ITACS is generally applicable to all extracellular ligand-ligand interactions for producing and selecting agents that bind to a cell-associated member of the ligand pair. The orphan NK cell receptors NKp30, NKp44, CD69 and NKp46, and the orphan CD83-molecule on dendritic cells are, however, particularly contemplated for use by ITACS. In fact, as described in Example 5 and FIG. 8, ITACS identified an antibody specific for NKp30L.

The use of fusion or hybrid proteins comprising at least the ligand-binding domain of, e.g., an orphan receptor and an Fc-domain of an antibody is a particular aspect of the invention. For example, such a fusion or hybrid protein can be used in steps (i) and/or (iv), facilitating detection of binding of the orphan receptor to target ligand-expressing cells (“target cells”) by, e.g., secondary antibodies against the Fc domain.

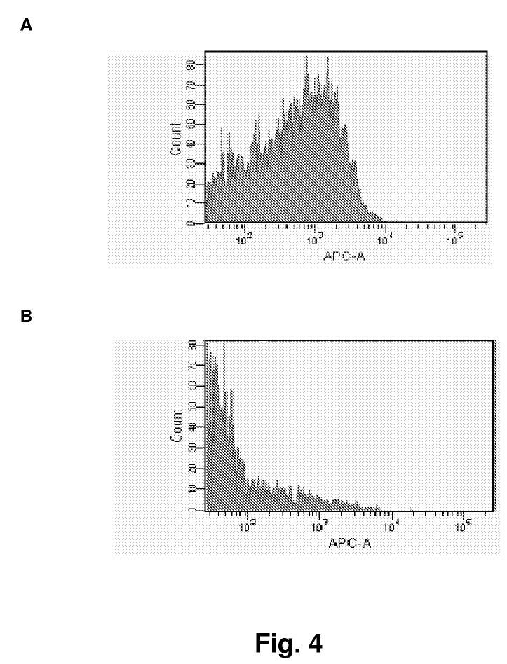

The novel orphan ligand-Fc constructs described herein can be used both as a therapeutic itself for targeting unknown ligands, and as a reagent for use in ITACS. The novel orphan ligand-Fc constructs comprise, in a particular aspect, a portion of the transmembrane region neighbouring the soluble, ligand-binding portion of the orphan ligand. This transmembrane-derived portion is herein referred to as a Flexible Transmembrane-Derived Linker (FTL). As shown in FIG. 4, an NKp30-Fc fusion protein designed in this manner (soINKp30-FTL-Fc) had improved ligand-binding characteristics as compared to prior art NKp30-Fc constructs. Thus, the invention provides fusion or hybrid proteins of NKp30 and other receptors incorporating a sequence normally found in the transmembrane region, in contrast to prior art constructs that have been truncated up-stream of the transmembrane region.

As described herein, the antibodies or other agents that have been identified by ITACS, or the fusion or hybrid proteins binding to ligands of orphan ligands, can be used therapeutically to treat conditions associated with the ligand-orphan ligand binding pair. Such conditions include, but are not limited to, cancer, autoimmune diseases, and viral infections. Also selecting for ‘depleting’ antibodies (i.e., antibodies or other agents capable of eliciting an ADCC or CDC response) may yield therapeutics suitable for the treatment of cancer and viral infections, whereas ‘non-depleting’ antibodies can be suitable for the treatment of autoimmunes diseases such as rheumatoid arthritis, multiple sclerosis, and Type I diabetes. In one aspect, in the case of antibodies, fusion proteins, or other agents targeting a ligand of an orphan NK cell activating receptor (e.g., NKp30, NKp44, or NKp46), such antibodies, fusion proteins, or other agents are characterized by their ability to reduce or inhibit NK cell-mediated lysis of target cells. Other applications for the identified antibodies or antibody fragments, or for the fusion or hybrid proteins described herein, include diagnostic applications to detect ligand expression, as well as methods of isolating and characterizing the unknown ligand.

Thus, the current invention provides a novel method for identifying agents that bind cell-associated ligands to orphan ligands, e.g., to orphan receptors such as NKp30 (SEQ ID NO:1), NKp44 (SEQ ID NO:2), NKp46 (SEQ ID NO:3); NKp80 (SEQ ID NO:13), CD83 (SEQ ID NO:14); and CD69 (SEQ ID NO:15). The invention also provides novel antibodies and fusion-proteins that bind such ligands, and the use of these as therapeutics to treat cancer and other diseases or disorders.

In one aspect, the present invention provides a method of identifying an antibody that binds to a cell surface-associated target ligand of an orphan ligand, which method comprises: (a) immunizing at least one vertebrate animal with a preparation of cells or cell membranes to which the orphan ligand binds; (b) preparing antibody-producing cells from the spleen of the vertebrate animal; and (c) selecting an antibody from an antibody-producing cell, which antibody competes with the orphan ligand in binding to the cells or cell membranes.

In another aspect, the present invention provides a method of identifying an antibody-producing cell that produces an antibody that binds to a cell surface-associated target ligand to an orphan ligand, which method comprises: (a) immunizing at least one vertebrate animal with a preparation of cells or cell membranes to which the orphan ligand binds; (b) preparing antibody-producing cells from spleens of the experimental animal; and (c) selecting any antibody-producing cell producing an antibody competing with the orphan ligand in binding to cells or cell membranes.

In a particular embodiment of any of the above methods, the selecting comprises (i) comparing the binding of an antibody from an antibody-producing cell to cells of the cell-line in the presence and absence of a reference protein selected from the group consisting of a full-length orphan ligand, a soluble portion of the orphan ligand, and a fusion or hybrid protein comprising a soluble portion of the orphan ligand, and (ii) identifying any antibody where the binding is lower in the presence of the reference protein than in the absence of the reference protein. In an alternative embodiment, the selecting comprises (i) comparing the binding of a reference protein selected from the group consisting of a full-length orphan ligand, a soluble portion of the orphan ligand, and a fusion or hybrid protein comprising a soluble portion of the orphan ligand to cells of the cell-line in the presence and absence of an antibody from an antibody-producing cell, and (ii) identifying any antibody where the binding is lower in the presence of the antibody than in the absence of the antibody. The fusion or hybrid protein in these embodiments may comprise a soluble portion of the orphan ligand associated or covalently bound to an antibody Fc domain, optionally via a linker. The fusion or hybrid protein may, also or alternatively, further comprise at least one amino acid residue of a transmembrane portion of the orphan ligand. The full-length orphan ligand or the soluble portion of the orphan ligand may be attached to a cell membrane or a solid support. In a particular embodiment, at least one of the reference protein and the antibody is labeled with a detectable moiety. For example, the detectable moiety may be a fluorescent, luminescent, or radioactive compound.

Optionally, the cells or cell membranes in (a) are from the same cell line as the cells or cell membranes in (c).

The antibody-producing cells in these methods can be, e.g., B cells or hybridoma cells. The antibody can be, for e.g., a murine or human antibody. The experimental animal can be, e.g., a mouse or rat.

In any of the above-described methods, the orphan ligand can be an orphan receptor. One type of orphan receptor contemplated is a receptor expressed on the surface of NK cells, such as, e.g., an NK cell activating receptor. In this embodiment, the orphan receptor can be selected from, e.g., NKp30, NKp44, NKp46, NKp80, and CD69. In another aspect, the orphan ligand is CD83.

In any of the above-described methods, the antibody selected in (c) may block the binding of the orphan ligand to the cell surface-associated ligand. Accordingly, the present invention provides for a method of identifying an antibody or antibody fragment that blocks the binding of a cell surface-associated ligand to an orphan ligand, which method comprises steps (a) to (c) of any of the preceding methods.

In another aspect, the present invention provides a method of identifying an antibody or antibody fragment that binds to a cell surface-associated target ligand of an orphan ligand, which method comprises: (a) providing a cell line to which the orphan ligand binds; (b) screening a library of antibodies or antibody fragments for an antibody competing with the orphan ligand in binding to the cells; and (c) selecting any antibody or antibody fragment competing with the orphan ligand. The library may be, e.g., a phage-display library.

In another aspect, the present invention provides a method of identifying an antibody that binds to a cell surface-associated target ligand of an NK cell receptor selected from NKp30, NKp44, and NKp46, which method comprises: (a) providing a cell line to the NK cell receptor binds; (b) immunizing at least one vertebrate animal with a preparation of cells or cell membranes of the cell line; (c) isolating B cells from the spleen of the at least one vertebrate animal; (d) preparing hybridomas from the isolated B cells; (e) evaluating the binding of an antibody from each hybridoma to cells of the cell line, in (i) the presence and (ii) the absence of a fusion or hybrid protein comprising a soluble portion of the NK cell receptor and an antibody Fc domain; and (f) selecting any antibody where the binding in (i) was lower than the binding in (ii).

The present invention also provides a method of identifying an antibody that binds to a cell surface-associated target ligand of an NK cell receptor selected from NKp30, NKp44, and NKp46, which method comprises: (a) providing a cell line to the NK cell receptor binds; (b) immunizing at least one vertebrate animal with a preparation of cells or cell membranes of the cell line; (c) isolating B cells from the spleen of the at least one vertebrate animal; (d) preparing hybridomas from the isolated B cells: (e) evaluating the binding of a fusion or hybrid protein comprising a soluble portion of the NK cell receptor and an antibody Fc domain to cells of the cell line in the presence of an antibody from each hybridoma; and (f) selecting any antibody from a hybridoma where the binding in is lower in the presence of the hybridoma than in the absence of any hybridoma. In one embodiment, the NK cell receptor is NKp30. In another embodiment, the fusion protein comprises the sequence of any of SEQ ID NOS:4, 5, and 6.

In another aspect, the present invention provides a method of identifying an agent that binds to NKp30L, which method comprises: (a) providing a plurality of test agents; (b) evaluating the binding of each test agent to a cell line expressing NKp30L in (i) the presence and (ii) the absence of a soluble NKp30-Fc fusion or hybrid protein comprising at least one amino acid residue from the transmembrane region of NKp30; and (c) selecting any test agent where the binding in (i) is lower than the binding in (ii) as an agent binding to NKp30L.

In another aspect, the present invention provides a method of identifying an agent that binds to NKp30L, which method comprises: (a) providing a plurality of test agents; (b) evaluating the binding of a soluble NKp30-Fc fusion or hybrid protein comprising at least one amino acid residue from the transmembrane region of NKp30 to a cell line expressing NKp30L in the presence of each test agent; and (c) selecting any test agent where the binding is lower in the presence of the test agent than in the absence of any test agent as an agent binding to NKp30L.

In another aspect, the present invention provides an antibody, antibody fragment, or agent identified according to the method of any of the preceding claims. In another aspect, the present invention provides a fragment or derivative of the antibody.

In another aspect, the present invention provides a fusion or hybrid protein comprising a soluble fragment of an NK cell receptor selected from NKp30, NKp44, and NKp46, covalently linked to an antibody Fc domain via a linker comprising at least one amino acid residue from the transmembrane region of the NK cell receptor. In one embodiment, the NK cell receptor is NKp30, and the fusion or hybrid protein comprises at least amino acid residues 139-149 of SEQ ID NO:1. In one aspect of this embodiment, the fusion protein comprises at least amino acid residues 20-138 of SEQ ID NO:1. In another aspect of this embodiment, the NKp30-Fc fusion protein comprises any of SEQ ID NOS:4 and 5. In another aspect of this embodiment, the NKp30-Fc fusion protein consists of any of SEQ ID NOS:4 and 5. In another embodiment, the NK cell receptor is NKp44, and the fusion or hybrid protein comprises at least amino acid residues 193-203 of SEQ ID NO:2. In another embodiment, the NK cell receptor is NKp46, and the fusion or hybrid protein comprises at least amino acid residue 256-266 of SEQ ID NO:3.

The present invention also provides a nucleic acid encoding such an identified antibody, a fusion protein or a soluble fragment to be used in preparing such a fusion protein, as well as expression vectors comprising such nucleic acids, host cells transformed with such vectors, and methods of producing such antibodies, fusion proteins, or soluble fragments by culturing such hosts cells under conditions allowing for expression of the antibodies, fusion proteins, or soluble fragments.

In another aspect, the present invention provides a method of inhibiting NK cell-mediated killing of a cell, the method comprising contacting the antibody, antibody fragment, antibody derivative, or agent identified by the methods described above, or the fusion or hybrid protein described above, which antibody, antibody fragment, antibody derivative, agent, or fusion or hybrid protein binds an NKp30L, NKp44L, or NKp46L, with a cell expressing NKp30L, NKp44L, or NKp46L.

In another aspect, the present invention provides for the use of the antibody, antibody fragment, antibody derivative, or agent identified by the methods described above, or the fusion or hybrid protein described above, for the preparation of a medicament to treat cancer or viral disease, wherein the antibody, antibody fragment, antibody derivative, agent, or fusion or hybrid protein is conjugated to a cytotoxic moiety or activates ADCC or CDC.

In another aspect, the present invention provides for the use of the antibody, antibody fragment, antibody derivative, or agent identified by the methods described above, or the fusion or hybrid protein described above, for the preparation of a medicament to treat an autoimmune disease.

In another aspect, the present invention provides for a method of treating cancer or a viral disease, the method comprising administering to a subject the antibody, antibody fragment, antibody derivative, or agent identified by the methods described above, or the fusion or hybrid protein described above, wherein the antibody, antibody fragment, antibody derivative, agent, or fusion or hybrid protein is conjugated to a cytotoxic moiety or activates ADCC or CDC. The cytotoxic moiety may, for example, be a toxin or a radioactive compound.

In another aspect, the present invention provides a method of treating an autoimmune disease, the method comprising administering to a subject the antibody, antibody fragment, antibody derivative, or agent identified by the methods described above, or the fusion or hybrid protein described above.

The following amino acid sequences are among those described in the accompanying Sequence Listing:

SEQ ID NO:1: Amino acid sequence of NKp30 (NCBI accession number NP667341).

SEQ ID NO:2: Amino acid sequence of NKp44.

SEQ ID NO:3: Amino acid sequence of NKp46.

SEQ ID NO:4: Amino acid sequence of soINKp30-FTL-hFc, made with a Flexible transmembrane Linker, and human IgG1 Fc.

SEQ ID NO:5: Amino acid sequence of soINKp30-FTL-mFc, made with a Flexible Transmembrane Linker, and murine IgG1 Fc.

SEQ ID NO:6: Amino acid sequence of soINKp30 (1L)-FTL-mFc, with mouse IgG1 Fc and having an extra leucine at the N-terminus as compared to the sequences in SEQ ID:1-3.

SEQ ID NO:7: Amino acid sequence of soINKp30-mFc, with mouse IgG1 Fc, made without a Flexible Transmembrane Linker but with a short linker (IEGRWMQ) instead.

SEQ ID NO:8: Amino acid sequence of soINKp30 (1L)-mFc, with mouse IgG1 Fc, made with an extra Leucine at the N-terminus, and without a Flexible Transmembrane Linker. SEQ ID NO:8 is identical to SEQ ID NO:7, apart from having the extra leucine.

SEQ ID NO:9: Amino acid sequence of soluble NKp30-hFc protein, made with N-terminal ALW and a 2 amino acid long linker between the NKp30 part and the human IgG1 Fc portion.

SEQ ID NO:10: Amino acid sequence of soluble NKp30-mFc protein, made with N-terminal ALW and a 2 amino acid long linker between the NKp30 part and the murine IgG1 Fc portion.

SEQ ID NO:11: Amino acid sequence of the NKp30 portion of a soluble NKp30-Fc protein available from R&D Systems Inc (catalog number 1849-NK).

SEQ ID NO:12: Amino acid sequence of a soluble NKp30-Fc protein described in WO2004053054.

SEQ ID NO:13: NKp80 amino acid sequence.

SEQ ID NO:14: CD83 amino acid sequence.

SEQ ID NO:15: CD69 amino acid sequence.

### Orphan Ligands

The present invention concerns, in part, a novel method for identifying hitherto unidentified ligands to receptors or other ligand-pair members. Such receptors or other ligand-pair members are herein referred to as “orphan ligands”, denoting that the other member(s) of the ligand-pair is/are unidentified. In one aspect, the unknown target ligands of any such ligand-pair are cell-associated. In another aspect, the orphan ligand naturally exists in a soluble form. In another aspect, the orphan ligand is an orphan cell-associated receptor. In another aspect, the orphan ligand is an orphan NK cell receptor. In another aspect, the orphan ligand is also or alternatively expressed on other cells of the immune system, such as, e.g., dendritic cells. Table 1 below lists exemplary orphan ligands suitable for application in the methods described in the present invention, along with the NCBI accession No. for the mRNA and/or protein sequence of the full-length orphan ligand and exemplary cell type(s) expressing the orphan ligand.

TABLE 1

| Orphan | NCBI accession | |
|---|---|---|
| Ligand | No. (SEQ ID NO) | Cells |
| NKp30 | NP_667341 | NK cells |
| | (SEQ ID NO:1) | |
| NKp44 | NP_004819 | NK cells |
| | (SEQ ID NO:2) | |
| NKp46 | NP_004820 | NK cells |
| | (SEQ ID NO:3) | |
| NKp80 | CAC29425 | NK cells |
| | (SEQ ID NO:13) | |
| CD83 | Z11697 | Dendritic |
| | (SEQ ID NO:14) | cells |
| CD69 | NP_001772 | NK cells |
| | (SEQ ID NO:15) | |

### Soluble Orphan Ligands

Soluble orphan ligands for use in the present invention typically comprise a fragment of at least an extracellular portion of the orphan ligand, or at least a fragment of a secreted orphan ligand, which fragment has been shown to bind specifically to cells expressing a target ligand of the orphan ligand. However, as described below, a soluble orphan ligand may also comprise one or more amino acids from the transmembrane region of a cell-associated orphan ligand. Alternatively, a soluble orphan ligand can exist in vivo in a soluble form, typically not associated with any cell-membrane.

A soluble fragment of a particular cell-membrane-associated orphan ligand can be known from the scientific literature or identified from, e.g., analysis of the amino acid sequence of the protein, using publicly available computer-based algorithms such as TMHMM (available at the world-wide web (www) address cbs.dtu.dk/services/TMHMM/), or determined by testing the ligand-binding capability of different fragments, as described in the Examples. Exemplary soluble fragments of NKp44, NKp46, NKp30, and CD83 (and/or Fc fusion or hybrid proteins comprising such soluble fragments) are also described in, e.g., WO2005051973, WO2005000086, WO0208287, WO2004053054, US2003219436, and Kunzendorf et al. (J Clin Invest 1996; 97:1204-10), all of which are hereby incorporated by reference in their entireties. An orphan ligand may also already naturally exist in a soluble state. Such soluble ligands include secreted proteins such as, e.g., cytokines.

Soluble fragments of orphan ligands can be produced by any known method of producing an amino acid-sequence, such as, e.g., controlled degradation of a purified protein by proteases or other chemical methods (Allen, Sequencing of proteins and peptides, 1989, Elsevier Science Publishers B.V.), recombinant expression of DNA encoding the soluble form, or chemical synthesis. Recombinant expression can be accomplished by transforming a host cell (e.g., a bacterial cell such as E. coli, or a mammalian cell such as CHO cells) with a vector containing a DNA sequence encoding a selected soluble fragment. General techniques for use in recombinant expression or other molecular biology applications described herein are known in the art (see, e.g., Sambrook et al., loc. cit., Ausubel, Current Protocols in Molecular Biology, Green Publishing Associates and Wiley Interscience, N.Y. (1989)). Chemical synthesis is commonly performed by coupling of the amino acid residues or peptide fragments to one another in correct order in liquid phase to produce the desired peptide. Another common strategy is the coupling of the amino acids to one another starting with a solid phase (resin) to which the C-terminal of the last amino acid of the sequence is coupled, whereupon the C-terminal of the penultimate amino acid is coupled to the N-terminal of the last amino acid, etc., finally releasing the built-up peptide from the solid phase (so called solid-phase technique).

In another aspect, the soluble orphan ligand or orphan receptor may be in the form of a dimer, generated by covalently coupling the C- or N-terminals of two soluble fragment monomers using a bivalent linker molecule. Suitable coupling agents or crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Co., Rockford, Ill. The soluble orphan ligand can also be a tetramer, generated by first biotinylating a soluble receptor monomer, followed by incubation of these with streptavidin.

Soluble receptors may also be in the form of soluble orphan ligand-IgG Fc fusion or hybrid proteins, or dimers of such soluble orphan ligand-IgG Fc fusion or hybrid proteins. Exemplary Fc fusion proteins have been described in the art, using either soluble fragments of a receptor or a soluble protein such as, e.g., a cytokine. An example of the latter is an IL-2 Fc fusion protein (Kunzendorf et al., J Clin Invest 1996; 97:1204-10).

However, whereas such soluble receptor-Fc fusion and hybrid proteins have long been known in the art, they often exhibit rather low binding avidity to their ligands, often resulting in difficult-to-detect binding to cells expressing their ligands. As described herein, the binding avidity can be improved by including a short polypeptide in-between the receptor and IgG Fc moieties. This short polypeptide can be any suitable peptide, selected to provide flexibility or proper conformation of the soluble fragment of the orphan ligand. In one aspect, this short polypeptide is derived from the N-terminal part of the transmembrane region of a soluble receptor such as NKp30. These transmembrane linkers are herein designated Flexible Transmembrane Linkers (FTLs). Other soluble receptors such as, but not limited to, NKp44, NKp46, and CD83, can also be expressed as IgG Fc fusion proteins, or prepared as IgG Fc hybrid proteins, with an FTL sequence inserted, resulting in avid binding to cells expressing the ligands of the receptor. In one aspect, the FTL comprises or consists of from 1 to 15 consecutive amino acids from the transmembrane region. In another aspect, the FTL comprises of consists of 1, 2, 3, 4, 5, 6, 7, 8, 9, 10, 11, 12, or more consecutive amino acids from the transmembrane region. In another aspect, the FTL comprises or consists of from 1 to 10 amino acids from the transmembrane region. In another aspect, the FTL comprises or consists of from 5 to 10 amino acid from the transmembrane region. In another aspect, the most C-terminal amino acid in the extracellular fragment of the orphan receptor is directly adjacent to the most N-terminal amino acid of the transmembrane portion of the receptor.

For example, in the case of NKp30, the FTL may comprise or consist of residues 139-149 of SEQ ID NO:1, where the most C-terminal amino acid of the extracellular region is residue 138 of SEQ ID NO:1. In separate embodiments, the FTL can comprise or consist of residues 139-148; 139-147; 139-146; 139-145; 139-144; 139-143; 139-142; and/or 139-141. Alternatively, the most C-terminal amino acid of the extracellular region is residue 139 of SEQ ID NO:1, and the FTL can comprise or consist of residues 140-149 of SEQ ID NO:1. In separate embodiments, the FTL can comprise or consists of residues 140-148; 140-147; 140-146; 140-145; 140-144; 140-143; 140-142; and/or 140-141.

In an additional or alternative aspect, the most N-terminal amino acid of the orphan ligand portion of an NKp30-Fc fusion or hybrid protein is residue 20 in SEQ ID NO:1, which is a tryptophan residue (Trp, or W). As described in Examples 3 and 5, such NKp30-Fc fusion proteins have a better ligand-binding ability than those including, e.g., residue 19 of SEQ ID NO:1 (leucine, Leu, or L).

In one embodiment, optimized NKp30-Fc proteins described herein, referred to as soINKp30-FTL-hFc or -mFc, are modified in both of the above aspects as compared to classical designs of Fc-fusion proteins. First, these constructs contain the Flexible Transmembrane Linker (FTL). Second, the predicted N-terminus of the mature protein, after removal of the signal sequence, is two residues downstream of the site predicted by computer algorithms, such that the N-terminus begins with WV (Trp-Val-). In exemplary NKp30-Fc fusion or hybrid proteins, the NKp30-portion thus comprises or consists of residues 20-149, 20-148, 20-147, 20-146, 20-145, 20-144, 20-143, 20-142, or 20-141 of SEQ ID NO:1.

In the case of NKp44, the FTL may comprise or consist of residues 193 (Val) to 203 (Ala) of SEQ ID NO:2. In separate aspects, the FTL comprises or consists of residues 193-202; 193-201; 193-200; 193-199; 193-198; 193-197; 193-196; and 193-195.

In the case of NKp46, the FTL may comprise or consists of residues 256 (Leu) to 266 (Leu) of SEQ ID NO:3. In separate aspects, the FTL comprises or consists of residues 256-265; 256-264; 256-263; 256-262; 256-261; 256-260; 256-259; and 256-258.

The present invention thus provides for IgG fusion or hybrid proteins of soluble receptor fragments, the fusion or hybrid protein comprising a soluble receptor fragment encompassing portions of both the extracellular region and the transmembrane region, and having improved binding properties as compared to a fragment which does not include any portion of a transmembrane region. These can be tested for binding activity in a similar manner as described in Examples 1-5. Example 1 also describes particular fusion proteins comprising a soluble portion of the NKp30 protein and a human or murine Fc domain.

In one aspect, the fusion or hybrid proteins comprises an additional amino acid residue between the orphan ligand and Fc-portions. While any suitable amino acid providing extra spacing and increased flexibility between the orphan ligand-portion and the Fc-portion may be used, exemplary amino acids include those that are relatively small and not heavily charged, such as alanine (A), used in constructs described in Example 1, and glycine (G). Other representative methods for producing and testing ligand-Fc fusion proteins can be found in WO2005051973, WO2005000086, WO0208287, WO2004053054, US2003219436, and Kunzendorf et al. (J Clin Invest 1996; 97:1204-10).

Various methods are available in the art to produce soluble receptor-Fc fusion or hybrid proteins. For example, a soluble portion of an orphan receptor, optionally comprising an FTL, can be linked to an FC polypeptide by, e.g., (1) chemical cross-linking; (2) affinity association by appending a moiety, such as a peptide, to soluble receptor segments and/or immunoglobulin polypeptide segments, and then joining the segments via the appended moiety or moieties to form a hybrid protein; and (3) linking soluble receptor segments and immunoglobulin polypeptide segments to form a single polypeptide chain via a polypeptide linker, i.e., a fusion protein.

In the first linkage category, any of a variety of conventional methods can be used to chemically couple (cross-link) two polypeptide chains. Covalent binding can be achieved either by direct condensation of existing side chains (e.g., the formation of disulfide bond between cysteine residues) or by the incorporation of external bridging molecules. Many bivalent or polyvalent agents are useful in coupling polypeptides.

In general, the cross-linking agents used are bifunctional agents reactive, e.g., with epsilon-amino group or thiol groups. These cross-linkers can be classified into two categories: homo- and hetero-bifunctional reagents. Homo-bifunctional reagents can react, e.g., with free thiols (e.g., generated upon reduction of disulfide bonds), and include, e.g., 5,5′-Dithiobis-(2-nitrobenzoic acid) (DNTB), and o-phenylenedimaleimide (O-PDM), which can form a thioether bond between two polypeptides having such free thiols. Hetero-bifunctional reagents can introduce a reactive group onto a polypeptide that will enable it to react with a second polypeptide. For example, N-succinimidyl-3-(2-pyridyldithio)propionate (SPDP) can react with a primary amino group to introduce a free thiol group. Other chemical cross-linking agents include, e.g., carbodiimides, diisocyanates, diazobenzenes, hexamethylene diamines, dimaleimide, glutaraldehyde, 4succinimidyl-oxycarbonyl-a-methyl a(2-pyridylthio)tolu-ene(SMPT) and N-succinimidyl-S-acetyl-thioacetate (SATA). Procedures for cross-linking polypeptides with such agents are well-known in the art. See, e.g., Pierce ImmunoTechnol-ogy Catalog & Handbook (1991) E8-E39; Karpovsky et al., J. Exp. Med. 1984; 160:1686 et seq.; Liu et al. Proc. Natl. Acad. Sci. 1985; 82:8648 et seq.; and U.S. Pat. No. 4,676,980.

Spacer arms between the two reactive groups of cross-linkers may have various lengths and chemical compositions. A longer spacer arm allows a better flexibility of the con-jugated polypeptides. while some particular components in the bridge (e.g., a benzene group) may lend extra stability to the reactive groups or an increased resistance of the chemical link to the action of various aspects (e.g., disulfide bond resistance to reducing re-agents). The use of peptide spacers such as FTLs or the peptide linkers or linker peptides described below is also contemplated.

In the second category of linkage methods, conventional methods can be used to append any of a variety of moieties (e.g., peptides) to soluble receptor portions and/or Fc polypeptides, thereby generating hybrid or fusion proteins which then can be associated via the appended moieties.

In one embodiment, moieties such as biotin and avidin (streptavidin) are bound or complexed to soluble receptor portions and/or immunoglobulin polypeptides, and these moieties interact to associate the two subunits.

In another embodiment, the appended moieties are both peptides, which may herein be referred to as “dimerization-promoting peptides.” Among the wide variety of such peptide linkers that can be used are the GST (glutathione S-transferase) fusion protein, or a dimerization motif thereof; a PDZ dimerization domain; FK-506 BP (binding protein) or a dimerization motif thereof; a natural or artificial helix-turn-helix dimerization domain of p53; and Protein A or its dimerization domain, domain B. In one embodiment, the appended peptides are components of a leucine zipper. The leucine zipper moieties are often taken from the human transcription factors c-jun and c-fos.

The dimerization-promoting peptide should provide an adequate degree of flexibility to prevent the two subunits from interfering with each other's activity, for example by steric hindrance, and to allow for proper protein folding. Therefore, it may be desirable to modify a dimerization-promoting peptide by altering its length, amino acid composition, and/or conformation, e.g., by appending to it still other “secondary linker moieties” or “hinge moieties.” The many types of secondary linker moieties include, e.g., tracts of small, preferably neutral and either polar or nonpolar, amino acids such. as, e.g., glycine, serine, threonine or alanine, at various lengths and combinations; polylysine; or the like. Alternatively, multiples of linkers and/or secondary linker moieties can be used. It is sometimes desirable to use a flexible hinge region, such as, e.g., the hinge region of human IgG, or polyglycine repeats interrupted by serine or threonine at certain intervals.

The length and composition of a dimerization-promoting peptide can readily be selected by one of skill in the art in order to optimize the desired properties of the soluble receptor, e.g., its ability to bind to its ligand.

The peptides can be appended to soluble receptor portions and immunoglobulin polypeptides by a variety of methods which will be evident to one of ordinary skill in the art, e.g., chemical coupling as described above (if necessary, following derivatization of appropriate amino acid groups); attachment via biotin/avidin interactions; covalent joining of the polypeptides by art-recognized methods (e.g., using appropriate enzymes); recombinant methods; or combinations thereof.

In the third linkage category, soluble receptor portions and/or immunoglobulin poly-peptides are covalently linked via a peptide linker. In this category, recombinant techniques are used to join soluble portions of each of two segments, in frame, to form a single chain polypeptide molecule. Preferably, the receptor portions are separated from one another by a linker peptide, of any length or amino acid composition, most preferably a flexible loop structure, which allows the two receptor moieties to lie at an appropriate distance from each other and in a proper alignment for optimal interaction. Typical linker peptides contain small, preferably neutral and either polar or nonpolar amino acids such as, e.g., glycine, serine, threonine or alanine, at various lengths and combinations; polylysine; or the like. The peptide linker can have at least one amino acid and may have 500 or more amino acids. Preferably, the linker is less than about 100 amino acids, more preferably about 2 to 30, most preferably about 3-10 amino acids. Flexible linker domains, such as the hinge region of human IgG, or polyglycine repeats interrupted by serine or threonine at certain intervals, can be used, alone or in combination with other moieties.

Recombinant methods which can be used to generate soluble receptor-Fc fusion proteins are conventional. Furthermore, assays described herein can be used to select linker peptides and to optimize parameters so that the optimal fragment of the orphan receptor, optionally comprising an FTL, is used in the constructs.

The herein described, or alternative formats of soluble receptors known in the art, can be prepared according to standard methods, and can be suitable for use in ITACS-screens or as therapeutic agents.

Soluble receptors may be used in ITACS-screens in which their binding is revealed using a secondary fluorochrome-conjugated secondary Ab which binds to the soluble orphan ligand. Alternatively, a fluorochrome (such as APC) may be directly attached to the soluble orphan ligand itself, eliminating the need for a secondary antibody. For example, a soluble orphan ligand can be conjugated to or otherwise stably associated with one or more fluorescent detection-facilitating agents (i.e., detection agents, tags, or labeling moieties) such as fluorescein, fluorescein isothiocyanate, rhodamine, 5-dimethylamine-1-napthalenesulfonyl chloride, lanthanide phosphors, and the like. Additional examples of suitable fluorescent labels include a ¹²⁵Eu label, an isothiocyanate label, a phycoerythrin label, a phycocyanin label, an allophycocyanin label, an o-phthaldehyde label, a fluorescamine label, etc. Examples of chemiluminescent labels include luminal labels, isoluminal labels, aromatic acridinium ester labels, imidazole labels, acridinium salt labels, oxalate ester labels, a luciferin labels, luciferase labels, aequorin labels, etc.

A soluble orphan ligand can also be labeled with enzymes or enzyme substrates that are useful for detection, such as horseradish peroxidase, β-galactosidase, luciferase, alkaline phosphatase, glucose oxidase, and the like. The orphan ligand can also be biotinylated, and detected through indirect measurement of avidin or streptavidin binding. Other labeling techniques include labeling with a predetermined polypeptide epitopes recognized by a secondary reporter (e.g., leucine zipper pair sequences, binding sites for secondary antibodies, metal binding domains, epitope tags, etc.). Additional examples of enzyme conjugate candidates include malate dehydrogenase, staphylococcal nuclease, delta-V-steroid isomerase, yeast alcohol dehydrogenase, α-glycerophosphate dehydrogenase, triose phosphate isomerase, asparaginase, glucose oxidase, ribonuclease, urease, catalase, glucose-6-phosphate dehydrogenase, glucoamylase, and acetylcholinesterase.

### Identification of Cell-Lines

Cell-lines expressing target ligands of orphan ligands can be identified by, e.g., flow cytometry analysis of cells stained with the soluble form of said orphan ligand, prepared as described above. For example, an assortment of cell-lines can be obtained from depositories such as American Type Culture Collection (ATCC), and the cell-lines screened for their ability to bind the soluble orphan ligand. In such a method, tumor-cells can be incubated with fixed amounts of fluorescently labeled soluble orphan ligand in tissue-culture medium containing 2% FCS, e.g. for 30 minutes, in the dark, on ice. After washing, the binding of soluble orphan ligand is analyzed by flow-cytometry. Alternatively, tumor-cells are incubated with non-fluorescently labeled soluble orphan ligand-Fc fusion protein comprising a human or murine Fc-domain, followed by incubation with fluorescently-labeled secondary antibodies that target the Fc-part of the fusion protein, prior to flow-cytometry analysis. In both assays, the binding of soluble orphan ligand to cells can be determined by analyzing the mean-fluorescence bound to individual cells, in comparison with the binding of either secondary antibodies alone, or a non-binding fluorescently labeled Fc-fusion protein. To confirm specificity, similar assays can be performed with soluble orphan ligand pre-incubated with a molar excess of antibodies against the orphan ligand known to inhibit binding of the orphan ligand. Cell-lines that are able to bind the soluble orphan ligand, but for which the binding can be competed with antibodies against the orphan ligand, are selected as cell-lines expressing the target ligand.

In an alternative exemplary assay, soluble receptor-Fc fusion proteins (e.g. NKp30-hFc) are used in flow-cytometry (e.g. FACS) to screen tumor cell-lines for cell-surface expressed ligands (e.g. NKp30L). For this, fixed amounts of tumor cells (e.g. K562, etc) are incubated on ice with various concentrations of soluble receptor-Fc fusion proteins which is conjugated to a fluorescent moiety (e.g. FITC, PE, APC, etc.). After incubation, the unbound soluble receptor-Fc fusion proteins are removed by washing the cells with PBS, and the binding of soluble receptor-Fc fusion-proteins to tumor-cells is analyzed by flow-cytometry (FACS).

Similar techniques based on alternative labeling and detection techniques (e.g., radioactive isotopes, avidin-biotin systems, or enzymatic detection methods), can be used according to similar principles.

### Preparation of Agents for Screening

Agent collections to be screened for binding to a target ligand to an orphan ligand include, but is not limited to, antibodies expressed by a collection of hybridomas, phage-display libraries or similar, and combinatorial libraries. Some of these agent collections are described below.

Once a suitable cell-line or cell-line(s) have been identified, these can be used to produce antibodies against the cell-lines, among them antibodies against the unidentified ligand. Various antibody production and purification techniques are known in the art and include those described in, e.g., Harlow and Lane: ANTIBODIES; A LABORATORY MANUAL, infra; Harlow and Lane: USING ANTIBODIES: A LABORATORY MANUAL (Cold Spring Harbor Laboratory Press (1999)); U.S. Pat. No. 4,376,110; and Ausubel et al, eds., CURRENT PROTOCOLS IN MOLECULAR BIOLOGY, Greene Publishing Assoc. and Wiley Interscience, N.Y., (1987, 1992). For example, monoclonal antibodies can be produced by the hybridoma method first described by Köhler et al., Nature, 256:495 (1975), or by other well-known, subsequently-developed methods (see, e.g., Goding, Monoclonal Antibodies: Principles and Practice, pp. 59-103 (Academic Press, 1986)). In such methods, animals are immunized with cells from a selected cell-line or a membrane preparation of cells from the selected cell-line (i.e., lysed cells prepared according to standard methods), B cells from the spleens of the animals are isolated, and hybridomas prepared. Hybridomas can be prepared by chemical fusion, electrical fusion, or any other suitable technique, with any suitable type of myeloma, heteromyeloma, or phoblastoid cell. Murine monoclonal antibodies can be obtained from immunization of mice. Monoclonal antibodies also can be obtained from hybridomas derived from antibody-expressing cells of other immunized non-human mammals such as rats, dogs, primates, etc. plasmacytoma, or other equivalent thereof and any suitable type of antibody-expressing cell.

Human antibodies can be generated in humanized transgenic animals (e.g., mice, rats, sheep, pigs, goats, cattle, horses, etc.) comprising human immunoglobulin loci and native immunoglobulin gene deletions, such as in a XenoMouse™ (Abgenix—Fremont, Calif., USA) (see, e.g., Green et al. Nature Genetics 7:13-21 (1994); Mendez et al. Nature Genetics 15:146-156 (1997); Green and Jakobovits J. Exp. Med. 188:483-495 (1998); European Patent No., EP 0 463 151 B1; International Patent Application Nos. WO 94/02602, WO 96/34096; WO 98/24893, WO 99/45031, WO 99/53049, and WO 00/037504; and U.S. Pat. Nos. 5,916,771, 5,939,598, 5,985,615, 5,998,209, 5,994,619, 6,075,181, 6,091,001, 6,114,598 and 6,130,364) or transgenic vertebrates comprising a minilocus of human Ig-encoding genes. Splenocytes from these transgenic mice or other vertebrates can be used to produce hybridomas that secrete human monoclonal antibodies according to well known techniques.

Preparing antibodies from an immunized animal includes obtaining B-cells, splenocytes, or lymphocytes from an immunized animal and using those cells to produce a hybridoma that expresses antibodies, as well as obtaining antibodies directly from the serum of an immunized animal. The isolation of splenocytes, e.g., from a non-human mammal is well-known in the art and, e.g., involves removing the spleen from an anesthetized non-human mammal, cutting it into small pieces and squeezing the splenocytes from the splenic capsule and through a nylon mesh of a cell strainer into an appropriate buffer so as to produce a single cell suspension. The cells are washed, centrifuged and resuspended in a buffer that lyses any red blood cells. The solution is again centrifuged and remaining lymphocytes in the pellet are finally resuspended in fresh buffer.

Once isolated and present in single cell suspension, the antibody-producing cells are fused to an immortal cell line. This is typically a mouse myeloma cell line, although many other immortal cell lines useful for creating hybridomas are known in the art. Preferred murine myeloma lines include, but are not limited to, those derived from MOPC-21 and MPC-11 mouse tumors available from the Salk Institute Cell Distribution Center, San Diego, Calif. U.S.A., X63 Ag8653 and SP-2 cells available from the American Type Culture Collection, Rockville, Md. U.S.A. The fusion is effected using polyethylene glycol or the like. The resulting hybridomas are then grown in selective media that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

The hybridomas can be grown on a feeder layer of macrophages. The macrophages are preferably from littermates of the non-human mammal used to isolate splenocytes and are typically primed with incomplete Freund's adjuvant or the like several days before plating the hybridomas. Fusion methods are described, e.g., in (Goding, “Monoclonal Antibodies: Principles and Practice,” pp. 59-103 (Academic Press, 1986)), the disclosure of which is herein incorporated by reference.

The cells are allowed to grow in the selection media for sufficient time for colony formation and antibody production. This is usually between 7 and 14 days. Hybridomas are then grown up in larger amounts in an appropriate medium, such as DMEM or RPMI-1640. Alternatively, the hybridoma cells can be grown in vivo as ascites tumors in an animal.

After sufficient growth to produce the desired monoclonal antibody, the growth media containing monoclonal antibody (or the ascites fluid) is separated away from the cells. The media can then be directly used in an ITACS procedure as described below, or purified according to known methods. Purification can typically achieved by gel electrophoresis, dialysis, chromatography using protein A or protein G-Sepharose, or an anti-mouse Ig linked to a solid support such as agarose or Sepharose beads (all described, for example, in the Antibody Purification Handbook, Amersham Biosciences, publication No. 18-1037-46, Edition AC, the disclosure of which is hereby incorporated by reference). The bound antibody is typically eluted from protein A/protein G columns by using low pH buffers (glycine or acetate buffers of pH 3.0 or less) with immediate neutralization of antibody-containing fractions. These fractions are pooled, dialyzed, and concentrated as needed.

In a typical method, mice are immunized with cells or a membrane preparation thereof from a cell-line expressing the unknown cell-surface ligand, identified as described above. The mice are immunized intraperitonally with, e.g., 0.1-10 million cells, 2×10⁶ cells, 1-100 μg membrane extract, or 20 μg membrane extract, which is typically repeated at intervals (e.g., bi-weekly, weekly, etc.). Immunizations with membrane extracts can be performed with Freund's Complete Adjuvant, whereas immunizations with cells can be injected in PBS alone. Mice can be immunized one to five times, or three times, in total, and are eye-bled about ten days after the final immunization to analyze the serum for antibodies responsive against the cells. Mice selected for generation of monoclonal antibodies can then be boosted i.v. with 10 μg membrane extract in PBS, whereas mice immunized with cells are usually not boosted prior to mAb production. Three days after boosting, spleens are harvested and used for hybridoma production. Spleen cells can be, for example, fused to FOX-NY myeloma cells by, e.g., PEG or electro-fusion techniques. The generated hybridoma cells are seeded into 24, 48, or 96 well tissue culture plates and the hybridomas or cell culture medium containing antibodies assayed according to ITACS as described below. Selected clones can be subjected to further rounds of subcloning and screening to establish stable hybridomas.

Transformed immortalized B cells (including human B cells) also can be used to produce antibodies, including human antibodies. Such cells can be produced by standard techniques, such as transformation with an Epstein Barr Virus, or a transforming gene. (See, e.g., “Continuously Proliferating Human Cell Lines Synthesizing Antibody of Predetermined Specificity,” Zurawaki, V. R. et al, in MONOCLONAL ANTIBODIES, ed. by Kennett R. H. et al, Plenum Press, N.Y. 1980, pp 19-33—text incorporated entirely).

Human antibodies or antibodies from other species, useful for ITACS screening, can also be generated through display-type technologies, including, without limitation, phage display, retroviral display, ribosomal display, and other related techniques, using methods well known in the art, and the resulting molecules can be subjected to additional maturation methods, such as affinity maturation, as such techniques also are well known (see, e.g., (Hoogenboom et al., J. Mol. Biol. 227: 381 (1991) (phage display); Vaughan, et al., Nature Biotech 14:309 (1996) (phage display); Hanes and Plucthau PNAS USA 94:4937-4942 (1997) (ribosomal display), Parmley and Smith Gene 73:305-318 (1988) (phage display), Scott TIBS 17:241-245 (1992), Cwirla et al. PNAS USA 87:6378-6382 (1990), Russel et al. Nucl. Acids Research 21:1081-1085 (1993), Hoogenboom et al. Immunol. Reviews 130:43-68 (1992), Chiswell and McCafferty TIBTECH 10:80-84 (1992), and U.S. Pat. No. 5,733,743). If display technologies are utilized to produce antibodies that are not human, such antibodies can be humanized, e.g., according to well-known methods.

Collections of antibodies and antibody fragments useful for ITACS screening can also be recovered from recombinant combinatorial antibody libraries, such as a scFv phage display library, which can be made with human VL and VH cDNAs prepared from mRNA derived from human lymphocytes. Methods for preparing and screening such libraries are known in the art. There are a number of commercially available kits for generating phage display libraries. There are also other methods and reagents that can be used in generating antibody display libraries (see, e.g., U.S. Pat. No. 5,223,409; PCT Publication Nos. WO 92/18619, WO 91/17271, WO 92/20791, WO 92/15679, WO 93/01288, WO 92/01047, and WO 92/09690; Fuchs et al. (1991) Bio/Technology 9:1370-1372; Hay et al. (1992) Hum. Antibod. Hybridomas 3:81-85; Huse et al. (1989) Science 246:1275-1281; McCafferty et al., Nature (1990) 348:552-554; Griffiths et al. (1993) EMBO J 12:725-734; Hawkins et al. (1992) J. Mol. Biol. 226:889-896; Clackson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc Acid Res 19:4133-4137; and Barbas et al. (1991) Proc. Natl. Acad. Sci. USA 88:7978-7982). Antibody libraries and methods of generating and using them are described in, e.g., International Patent Application WO 92/01047, McCafferty et al., Nature (1990) 348:552-554; U.S. Pat. Nos. 5,969,108; 5,872,215; 5,871,907; 5,858,657; and Griffiths et al., (1993) EMBO J 12:725-734).

Other agents than antibodies can also be screened for their ability to bind a ligand of an orphan ligand, using an approach similar to ITACS where competition with, e.g., a fusion protein of a soluble form of the orphan ligand is used. Such agents can be found, e.g., in a combinatorial chemical library. A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37: 487-493 (1991) and Houghton et al., Nature 354: 84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90: 6909-6913 (1993)), vinylogous polypeptides (Hagihara et al., J. Amer. Chem. Soc. 114: 6568 (1992)), nonpeptidal peptidomimetics with glucose scaffolding (Hirschmann et al., J. Amer. Chem. Soc. 114: 9217-9218 (1992)), analogous organic syntheses of small compound libraries (Chen et al., J. Amer. Chem. Soc. 116: 2661 (1994)), oligocarbamates (Cho et al., Science 261: 1303 (1993)), and/or peptidyl phosphonates (Campbell et al., J. Org. Chem. 59: 658 (1994)), nucleic acid libraries (see Ausubel, Berger and Sambrook, all supra), peptide nucleic acid libraries (see, e.g. U.S. Pat. No. 5,539,083), antibody libraries (see, e.g., Vaughn et al., Nature Biotechnology, 14(3): 309-314 (1996) and PCT/US96/10287), carbohydrate libraries (see, e.g., Liang et al., Science, 274: 1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

### ITACS

An ITACS procedure is a convenient method to identify agents that bind to, e.g., a target ligand of an another ligand such as, e.g., an orphan receptor. While the method is especially useful to identify ligands to orphan receptors, the same principles can be applied to other members of ligand pairs which are not receptors and/or not orphan.

The following sections describe some non-limiting features of different steps of ITACS. It is to be understood that, depending on the particular ligand-ligand pair, and the agent collection or library screened, the ITACS procedure can be modified for optimal performance on a case-by-case basis, and is not limited to the specific exemplary method steps described here.

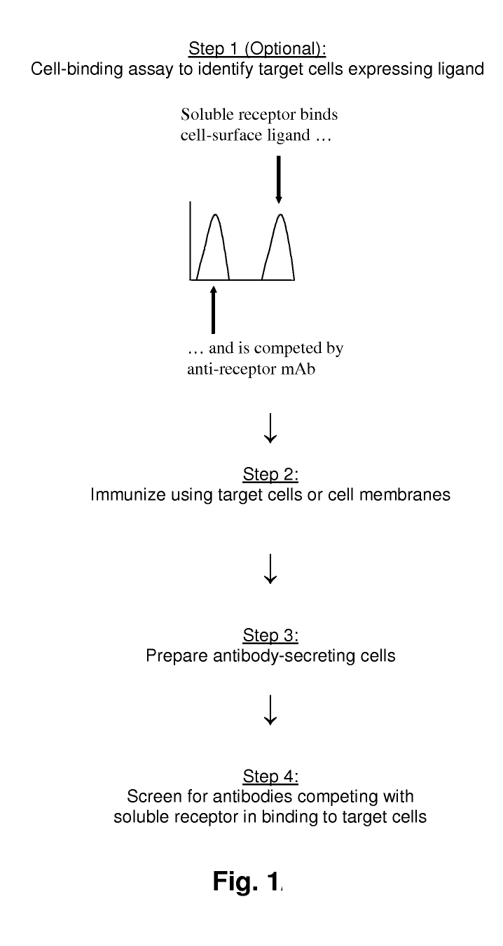

An exemplary ITACS procedure is outlined in FIG. 1. The identified agents are identified and characterized by their ability to interfere with, reduce, and/or block the binding of a soluble portion of the orphan ligand to the target ligand.

As described above, in one aspect, the agent collection to be screened is a collection of hybridomas, B-cells, or other antibody-producing cells, producing antibodies against various epitopes on a cell-line to which the soluble orphan ligand or receptor binds (steps 1 to 3 of FIG. 1). In the subsequent ITACS step (step 4 of FIG. 1), the antibodies from the antibody-producing cells are incubated with cells to which the orphan ligand binds in the presence of a soluble portion of the orphan ligand. In this step, antibodies competing with the soluble receptor in binding to the cells are identified. In this context, “competing” means that the presence of an antibody reduces the binding of the soluble receptor to the cells as compared to the binding of the soluble receptor to the cells in the absence of antibody. For example, an antibody identified as competing with the orphan receptor may reduce the binding of the soluble receptor with at least about 10%, at least about 20%, at least about 30%, at least about 40%, at least about 50%, at least about 60%, at least about 70%, at least about 80%, or at least about 90%. In a particular aspect, the antibody reduces the binding of the orphan receptor by at least 25%. The soluble receptor can be labeled to detect binding as described elsewhere herein. Alternatively, the soluble receptor may be in the form of a fusion or hybrid protein of an IgG Fc domain, which then can be tagged using secondary antibodies against the Fc portion, followed by detection of the secondary antibodies.

In an alternative or additional aspect, the ITACS step may comprise screening for antibodies where the presence of the soluble receptor reduces the binding of an antibody to the cells, as compared to the binding of the antibody to the cells in the absence of soluble receptor. An antibody identified as competing with the soluble receptor can, in this aspect, be identified as an antibody whose binding to the cells is reduced by at least about 10%, at least about 20%, at least about 30%, at least about 40%, at least about 50%, at least about 60%, at least about 70%, at least about 80%, or at least about 90%, in the presence of the soluble receptor. In a particular aspect, the antibody reduces the binding of the orphan receptor by at least 25%. The antibody can either be suitable labeled to detect binding, as described elsewhere herein, or detected using secondary antibodies (e.g., if screening a collection of human antibodies their cell-binding can be detected using mouse-anti-human antibodies).

Various experimental set-ups of the competition step can be used. Typically, the competition step takes place in a test vial, such as a well, in the presence of antibody, soluble receptor, and cells to which the soluble receptor binds. The cells to which the orphan ligand bind can be attached to a solid surface in the test vial, such as the bottom of a well or to a bead, via normal cell-surface interaction mechanisms or other means. Cells in suspension or cell membrane preparations may also be used in connection with a suitable method of separating the cells from unbound labeled binding agent after incubation. In various aspects, the antibody can be provided via addition of antibody-producing cell, culture supernatant of an antibody-producing cell, or a purified preparation of an antibody, to the test vial. The reagents are then incubated for a suitable period of time and at a suitable temperature to allow for soluble receptor and (if applicable) antibody binding to the cells. The cells are then optionally washed one or more times before evaluating the amount of soluble receptor or antibody bound to the cells. The amount of cell-bound soluble receptor/antibody is then compared to, e.g., the amount of cell-bound soluble receptor/antibody in the absence of antibody/receptor, or another suitable control value. Depending on the assay, unspecific binding of a soluble receptor or antibody can be corrected for by use of similar compounds which do not bind to the cells, or by antibodies against the soluble receptor, preventing its binding to the target ligand.

In an exemplary assay, hybridoma supernatants are screened for the presence of anti-ligand mAbs by pre-incubating cells with these supernatants followed by addition of the soluble orphan-receptor. Hybridoma supernatants that result in reduced binding of the soluble orhpan receptor are designated “positive clones”. The analysis of stained cells can be performed by, e.g., flow cytometry using a FACSarray, or by Fmat (PE Biosystems, CA).

In another exemplary assay, antibodies that target NKp30L are identified by flow-cytometry (e.g. FACS, FACSarray) or Fmat, in a competition assay in which antibodies are screened for their capacity to prevent the binding of soINKp30-hFc to NKp30L-expressing tumor cell-lines (e.g. K562). For this, tissue-culture supernatants from monoclonal B-cell-derived hybridoma's, derived from mice immunized with NKp30L-expressing tumor-cells (e.g. K562), are incubated with fixed amounts of NKp30L-expressing tumor-cells (e.g 104 K562 or HEK293 cells), for 30-60 minutes on ice. Subsequently, a fixed amount of fluorescently labeled soINKp30-hFc is added to each incubation mixture (e.g. 0.1 μg/ml APC-soINKp30-hFc), which is then incubated for another 30-60 minutes on ice. After incubation, cells are washed to remove unbound proteins, and binding of soINKp30-hFc to cells is analyzed by flow-cytometry or Fmat. In both assays, soINKp30-hFc binding to cells is determined by analyzing the mean fluorescence of individual cells. In the assay, antibodies are considered NKp30L-binding antibodies when they reduce or prevent soINKp30-hFc-binding to tumor-cells in these assays, in comparison with the binding of soINKp30-hFc to tumor-cells which have not been pre-incubated with tissue-culture supernatants from hybridomas.

Once an antibody (or, in the case of phage-display and combinatorial libraries, an antibody fragment or small molecule) binding to the ligand of the orphan ligand, thereby blocking ligand-ligand interaction, has been identified, larger amounts of antibody, antibody fragment, or small molecule can be produced, purified, and modified according to known techniques, if desired. For example, nucleic acid sequences encoding an antibody or antibody fragment can be retrieved, allowing for recombinant production of the antibodies or fragments in host cells, according to conventional methods. The antibody, antibody fragment, or small molecule can also be tested for its efficacy in treating a condition associated with the ligand-orphan receptor pair, such as cancer or an autoimmune disease.

As described in the sections above, Fmat equipment is suitable for the ITACS screening competition step, whether screening antibodies or agents from combinatorial libraries. Fmat, an abbreviation for Fluorimetric Microvolume Assay Technology, can be used for quantitative determination of receptor-ligand interactions using a scanner designed to perform high-throughput screening assays in multiwell plates with no wash steps (Mellentin-Michelotti et al., Anal Biochem. 1999; 272:182-190). Various Fmat assay formats that can be adapted for use in ITACS are described in the literature (e.g., Mellentin-Michelotti et al., supra; Swartzman et al., Anal Biochem. 1999; 271:143-151; Lee et al., J Biomolecular Screening 2003; 81-88). Other high-throughput screening techniques that can be adapted for use in ITACS include, but are not limited to, Biacore (using, e.g., cell lysates or cell membranes), cell-based ELISA (using, e.g., intact cells or cell membranes), and various antibody microarray formats known in the art (reviewed by Glokler and Angenendt, J Chromatograph B. 2003; 797:229-240, and Biacore technology (see, e.g., Zhukov et al., J Biomol Techniques 2004; 15:112-119). In addition, flow cytometry may be used, especially in medium- or high-throughput formats, using, e.g., a FACSarray (Beckton Dickinson, Calif.) or similar.

Similar competition assays can be designed for screening phage-display libraries and combinatorial libraries. For example, cells or tissue-sections can be immobilized in an ELISA-plate and used for panning of a phage-display library. Phages binding in the ELISA, which display competition with soluble receptor-Fc protein, are specific for the orphan-ligand.

Once one or more antibodies have been identified, nucleic acids encoding the antibodies can be retrieved from the hybridomas or other antibody-producing cells, and the antibodies produced by recombinant techniques according to conventional methods in the art.

### Antibody Fragments and Derivatives

An identified antibody or antibody fragment can be modified, e.g., to produce alternative antibodies or fragments, antibody fragments, and/or to derivatize the antibody or antibody fragment with another compound. The following sections exemplify various modifications than can be made.

If desired, the class of an antibody obtained by antibody producing cells may be “switched” by known methods. For example, an antibody that was originally produced as an IgM molecule may be class switched to an IgG antibody. Class switching techniques also may be used to convert one IgG subclass to another, e.g., from IgG1 to IgG2. Thus, the effector function of the antibodies of the invention may be changed by isotype switching to, e.g., an IgG1, IgG2, IgG3, IgG4, IgD, IgA, IgE, or IgM antibody for various therapeutic uses.

For example, in therapeutic applications where it is desirable to reduce the number of cells expressing a ligand of an orphan ligand (e.g., where the ligand is overexpressed on cancer cells), the antibody or antibody derivative can have an Fc-portion that activates antibody-dependent cellular cytotoxicity (ADCC) or cellular-dependent cytotoxicity (CDC).

Chimeric antibodies may be produced by recombinant processes well known in the art (see, e.g., Cabilly et al, Proc. Natl. Acad. Sci. USA 81:3273-3277 (1984); Morrison et al., Proc. Natl. Acad. Sci. USA 81:6851-6855 (1984); Boulianne et al., Nature 312:643-646 (1984); European Patent Application 125023; Neuberger et al., Nature 314:268-270 (1985); European Patent Application 171496; European Patent Application 173494; WO 86/01533; European Patent Application 184187; Sahagan et al., J. Immunol. 137:1066-1074 (1986); Robinson et al., International Patent Publication #PCT/US86/02269 (published May 7, 1987); Liu et al., Proc. Natl. Acad. Sci. USA 84:3439-3443 (1987); Sun et al., Proc. Natl. Acad. Sci. USA 84:214-218 (1987); and Better et al., Science 240:1041-1043 (1988)). For example, an identified murine monoclonal antibody can be chimerized to an antibody having constant domains from a human monoclonal antibody.

Humanized monoclonal antibodies or murine antibodies or antibodies from other non-human species can also be made. A “humanized” antibody is an antibody that is derived from a non-human species, in which certain amino acids in the framework and constant domains of the heavy and light chains have been mutated so as to avoid or abrogate an immune response in humans. For further details regarding the characteristics and production of typical humanized antibodies, see, e.g., Jones et al., Nature 321:522-525 (1986); Riechmann et al., Nature 332:323-329 (1988); and Presta, Curr. Op. Struct. Biol. 2:593-596 (1992). A humanized antibody may also be produced by fusing the constant domains from a human antibody to the variable domains of a non-human species. Examples of methods that can be used to make humanized antibodies may be found in, e.g., U.S. Pat. Nos. 6,054,297, 5,886,152, and 5,877,293. Humanization can be essentially performed following the method of Winter and co-workers (see, e.g., Jones et al., Nature 321:522-525 (1986); Riechmann et al., Nature 332:323-327 (1988); Verhoeyen, et al., Science, 239:1534-1536 (1988)), by substituting rodent complementarity determining regions (“CDRs”) or CDR sequences for the corresponding sequences of a human antibody. Accordingly, in such humanized antibodies, the CDR portions of the human variable domain are substituted by the corresponding sequence from a non-human species. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (see, e.g., Sims et al., J. Immunol., 151:2296 (1993) and Chothia et al., J. Mol. Biol., 196:901 (1987) for a description of such methods and related principles). Another method uses a particular framework derived from the consensus sequence of all human antibodies of a particular subgroup of light or heavy chains. The same framework may be used for several different humanized antibodies (see, e.g., Carter et al., Proc. Natl. Acad. Sci. USA, 89:4285 (1992) and Presta et al., J. Immunol., 151:2623 (1993)).

Murine antibodies or antibodies from other species can be humanized or primatized using any suitable techniques, a number of suitable techniques being already well known in the art (see e.g., Winter and Harris Immunol Today 14:43-46 (1993) and Wright et al. (Crit. Reviews in Immunol. 12125-168 (1992)). The antibody of interest may be engineered by recombinant DNA techniques to substitute the CH1, CH2, CH3, hinge domains, and/or the framework domain with the corresponding human sequence (see WO 92/02190 and U.S. Pat. Nos. 5,530,101, 5,585,089, 5,693,761, 5,693,792, 5,714,350, and 5,777,085). Also, the use of 1 g cDNA for construction of chimeric immunoglobulin genes is known in the art (see, e.g., Liu et al. P.N.A.S. 84:3439 (1987) and J. Immunol. 139:3521 (1987)). mRNA can be isolated from a hybridoma or other cell producing the antibody and used to produce cDNA. The cDNA of interest may be amplified by the polymerase chain reaction (PCR) using specific primers (U.S. Pat. Nos. 4,683,195 and 4,683,202). Alternatively, a library can be made and screened to isolate a sequence of interest. The nucleic acid sequence encoding the variable region of the antibody can then fused to human constant region sequences. Sequences of human constant regions (as well as variable regions) may be found in Kabat et al. (1991) Sequences of Proteins of Immunological Interest, N.I.H. publication no. 91-3242 and more recent and related data can be accessed at World Wide Web (www) address .biochem.ucl.ac.uk/˜martin/abs/GeneralInfo.html. The choice of isotype for a designed antibody typically can be guided by the desired effector functions, such as complement fixation, or activity in antibody-dependent cellular cytotoxicity. Exemplary isotypes are IgG1, IgG2, IgG3, and IgG4. Either of the human light chain constant regions, kappa or lambda, may be used. A humanized antibody encoded by such a nucleic acid can then be expressed by conventional methods.

In addition to such antibody-like molecules and full-sized antibodies, “fragments” of the identified antibodies can be made. Antibody “fragments” that retain/exhibit the ability to specifically bind to the ligand of the orphah ligand may generally be obtained by any known technique, such as, but not limited to, enzymatic cleavage, peptide synthesis, and recombinant protein production techniques. Examples of antibody fragments include (i) a Fab fragment, a monovalent fragment consisting essentially of the VL, VH, CL and CH I domains; (ii) F(ab)₂ and F(ab′)2 fragments, bivalent fragments comprising two Fab fragments linked by a disulfide bridge at the hinge region; (iii) a Fd fragment consisting essentially of the VH and CH1 domains; (iv) a Fv fragment consisting essentially of the VL and VH domains of a single arm of an antibody, (v) a dAb fragment (Ward et al., (1989) Nature 341:544-546), which consists essentially of a VH domain; and (vi) one or more isolated CDRs or a functional paratope. Additional antibody fragments include Fab′ fragments, dsFv molecules, diabodies, and the like.

In one exemplary aspect, the invention provides an antibody fragment comprising a first polypeptide chain that comprises any of the heavy chain CDRs described herein and a second polypeptide chain that comprises any of the light chain CDRs described herein, wherein the two polypeptide chains are covalently linked by one or more interchain disulfide bonds. In a more particular aspect, the invention provides a two-chain antibody fragment having such features wherein the antibody fragment is selected from Fab, Fab′, Fab′--SH, Fv, and/or F(ab′)2 fragments. Other antibody “fragments” include “kappa bodies” (see, e.g., III et al., Protein Eng 10: 949-57 (1997)) and “janusins” (described further elsewhere herein).

Antibodies can be fragmented using conventional techniques, and the fragments screened for utility in the same manner as described above for whole antibodies. For example, F(ab′)2 fragments can be generated by treating antibody with pepsin. The resulting F(ab′)2 fragment can be treated to reduce disulfide bridges to produce Fab′ fragments. Fab fragments can be obtained by treating an IgG antibody with papain; F(ab′) fragments can be obtained with pepsin digestion of IgG antibody. A F(ab′) fragment also can be produced by binding Fab′ described below via a thioether bond or a disulfide bond. A Fab′ fragment is an antibody fragment obtained by cutting a disulfide bond of the hinge region of the F(ab′)2. A Fab′ fragment can be obtained by treating a F(ab′)2 fragment with a reducing agent, such as dithiothreitol. Antibody fragment peptides can also be generated by expression of nucleic acids encoding such peptides in recombinant cells (see, e.g., Evans et al., J. Immunol. Meth. 184: 123-38 (1995)). For example, a chimeric gene encoding a portion of a F(ab′)2 fragment can include DNA sequences encoding the CH1 domain and hinge region of the H chain, followed by a translational stop codon to yield such a truncated antibody fragment molecule.

Although the two domains of the Fv fragment, VL and VH, are coded for by separate genes, they can be joined, e.g., using recombinant methods, by a synthetic and typically flexible linker that enables them to be made as a single protein chain in which the VL and VH regions (typically the heavy and light chains in the Fv region of an antibody) pair to form monovalent molecules (known as single chain antibodies or single chain Fv (scFv) molecules—see e.g., Bird et al. (1988) Science 242:423-426: and Huston et al. (1988) Proc. Natl. Acad. Sci. USA 85:5879-5883). Usually the flexible linker is of about 10, 12, 15, or more amino acid residues in length. Methods of producing such antibodies are described in, e.g., U.S. Pat. No. 4,946,778; THE PHARMACOLOGY OF MONOCLONAL ANTIBODIES, vol. 113, Rosenburg and Moore eds. Springer-Verlag, New York, pp. 269-315 (1994), Bird et al. (1988) Science 242:423-426; Huston et al. (1988) Proc. Natl. Acad. Sci. USA 85:5879-5883; and McCafferty et al., Nature (1990) 348:552-554. A single chain antibody may be monovalent, if only a single VH and VL are used, bivalent, if two VH and VL are used, or polyvalent, if more than two VH and VL are used to form the antibody.

Diabodies are bivalent, bispecific antibodies in which VH and VL domains are expressed on a single polypeptide chain, but using a linker that typically is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). A diabody can be considered an antibody fragment in which scFvs having the same or different antigen binding specificity form a dimer and, accordingly, is a molecule that has a divalent antigen binding activity to the same antigen or to two different antigens. Diabodies are described more fully in, for example, EP 404,097 and WO 93/11161.

A dsFV molecule can be obtained by binding polypeptides in which one amino acid residue of each of VH and VL is substituted with a cysteine residue via a disulfide bond between the cysteine residues. The amino acid residue which is substituted with a cysteine residue can be selected based on a three-dimensional structure estimation of the antibody, e.g., in accordance with the method described by Reiter et al. (Protein Engineering, 7, 697 (1994)). Linear antibodies, which comprise a pair of tandem Fd segments that form a pair of antigen binding regions (such antibodies can be bispecific or monospecific). Linear antibodies are more fully described in, e.g., Zapata et al. Protein Eng. 8(10):1057-1062 (1995).

A “variant” antibody is an antibody that differs from a parent antibody by one or more suitable amino acid residue substitutions, deletions, insertions, or terminal sequence additions in at least the CDRs or other VH and/or VL sequences (provided that at least a substantial amount of the epitope binding characteristics of the parent antibody are retained, if not improved upon, by such changes). Thus, for example, in an antibody variant or antibody-like peptide variant, one or more amino acid residues can be introduced or inserted in or adjacent to one or more of the hypervariable regions of a parent antibody, such as in one or more CDRs. For example, an antibody variant can comprise about 1-30 inserted amino acid residues, but about 2-10 inserted amino acid residues is more typically suitable. Amino acid sequence variants of the antibody can be obtained by, for example, introducing appropriate nucleotide changes into an antibody-encoding nucleic acid (e.g., by site directed mutagenesis), by chemical peptide synthesis, or any other suitable technique. Such variants include, for example, variants differing by deletions from, and/or insertions into and/or substitutions of, residues within the amino acid sequences of the identified antibody. A variation in a framework region or constant domain may also be made to alter the immunogenicity of the variant antibody with respect to the parent antibody, to provide a site for covalent or non-covalent binding to another molecule, or to alter such properties as complement fixation. Variations in an antibody variant may be made in each of the framework regions, the constant domain, and/or the variable regions (or any one or more CDRs thereof) in a single variant antibody. Alternatively, variations may be made in only one of the framework regions, the variable regions (or single CDR thereof), or the constant domain in an antibody. Alanine scanning mutagenesis techniques, such as described by Cunningham and Wells (1989), Science 244:1081-1085, can be used to identify suitable residues for substitution or deletion in generating variant VL, VH, or particular CDR sequences, although other suitable mutagenesis techniques also can be applied. Multiple amino acid substitutions also can be made and tested using known methods of mutagenesis and screening, such as those disclosed by Reidhaar-Olson and Sauer, Science 241:53-57 (1988) or Bowie and Sauer Proc. Natl. Acad. Sci. USA 86:2152-2156 (1989). Additional techniques that can be used to generate variant antibodies include the directed evolution and other variant generation techniques described in, e.g., US 20040009498; Marks et al., Methods Mol. Biol. 2004; 248:327-43 (2004); Azriel-Rosenfeld et al., J Mol. Biol. 2004 Jan. 2; 335(1):177-92; Park et al., Biochem Biophys Res Commun. 2000 Aug. 28; 275(2):553-7; Kang et al., Proc Natl Acad Sci USA. 1991 Dec. 15; 88(24):11120-3; Zahnd et al., J Biol. Chem. 2004 Apr. 30; 279(18):18870-7; Xu et al., Chem. Biol. 2002 August; 9(8):933-42; Border et al., Proc Natl Acad Sci USA. 2000 Sep. 26; 97(20):10701-5; Crameri et al., Nat. Med. 1996 January; 2(1):100-2; and as more generally described in, e.g., International Patent Application WO 03/048185.

A specific type of variant antibody is bispecific antibodies. These can be produced by variety of known methods including fusion of hybridomas or linking of Fab′ fragments (see, e.g., Songsivilai & Lachmann Clin. Exp. Immunol. 79: 315-321 (1990) and Kostelny et al. J. Immunol. 148:1547-1553 (1992)). Traditionally, the recombinant production of bispecific antibodies is based on the co-expression of two immunoglobulin heavy chain-light chain pairs, where the two heavy chains have different specificities (see, e.g., Milstein and Cuello, Nature, 305: 537 (1983)). Because of the typical random assortment of immunoglobulin heavy and light chains, these hybridomas (quadromas) produce a potential mixture of 10 different antibody molecules, of which only one typically has the desired bispecific structure. The purification of the correct molecule, which is usually done by affinity chromatography, although suitable, can be rather cumbersome, and the product yields can be relatively low. Similar procedures are disclosed in WO 93/08829 and Traunecker et al., EMBO J., 10: 3655 (1991). According to a different approach, antibody variable domains with the desired binding specificities (antibody-antigen combining sites) are fused to immunoglobulin constant domain sequences by recombinant or synthetic methods. The variable domain sequence is typically fused to an immunoglobulin heavy chain constant domain, comprising at least part of the hinge, CH2, and CH3 regions. A first heavy-chain constant region (CH1), containing the site necessary for light chain binding, also typically is present in at least one of the fusion peptides. In a more specific example of this type of approach, a bispecific antibody is produced comprising a hybrid immunoglobulin heavy chain with a first binding specificity in one arm, and a hybrid immunoglobulin heavy chain-light chain pair (providing a second binding specificity) in the other arm. Such an asymmetric structure can facilitate the separation of the desired bispecific compound from unwanted immunoglobulin chain combinations (such an approach is described in WO 94/04690). For further description of related methods for generating bispecific antibodies see, for example, Suresh et al., Methods in Enzymology, 121:210 (1986).

Cross-linked or “heteroconjugate” antibodies are another type of bispecific antibody provided by the invention. Derivatives of such antibodies also can be advantageous for certain applications. For example, one of the antibodies in a heteroconjugate can be coupled to avidin and the other to biotin. Such antibodies have, for example, been proposed to target immune system cells to unwanted cells (see, e.g., U.S. Pat. No. 4,676,980). Heteroconjugate antibodies may be made using any convenient cross-linking methods. Suitable peptide cross-linking agents and techniques are well known in the art, and examples of such agents and techniques are disclosed in, e.g., U.S. Pat. No. 4,676,980.

Bispecific antibodies and antibody-like molecules (e.g., bispecific molecules generated from two antibody fragments) generally can be prepared using chemical linkage techniques. Brennan et al., Science, 229: 81 (1985), for example, describe a procedure wherein intact antibodies are proteolytically cleaved to generate F(ab′)2 fragments. These fragments may then be reduced in the presence of the dithiol complexing agent sodium arsenite to stabilize vicinal dithiols and prevent intermolecular disulfide formation. The Fab′ fragments generated can then converted to thionitrobenzoate (TNB) derivatives. One of the Fab′-TNB derivatives can then be reconverted to the Fab′-thiol by reduction with mercaptoethylamine and mixed with an equimolar amount of the other Fab′-TNB derivative to form a bispecific antibody.

Fab′-SH fragments also recovered from E. coli also can be chemically coupled to form bispecific antibodies. Shalaby et al., J. Exp. Med., 175: 217-225 (1992), for example, describe the production of a fully humanized bispecific antibody F(ab′)2 molecule, according to a related technique.

Various techniques for making and isolating bispecific antibody fragment molecules directly from recombinant cell culture have also been described. For example, bispecific antibodies have been produced using leucine zippers (see, e.g., Kostelny et al., J. Immunol., 148(5): 1547-1553 (1992)). The “diabody” technology described by Hollinger et al., Proc. Natl. Acad. Sci. USA, 90:6444-6448 (1993) also has provided an alternative mechanism for making bispecific antibody fragment molecules (see also Alt et al., FEBS Letters, 454 (1990) 90-94 for a description of similar diabody-related techniques). Another strategy for making bispecific antibody fragment molecules by the use of single-chain Fv (sFv) dimers has also been reported. See, e.g., Gruber et al., J. Immunol., 152:5368 (1994). In addition, bispecific antibodies may be formed as “Janusins” (Traunecker et al., EMBO J 10:3655-3659 (1991) and Traunecker et al., Int J Cancer Suppl 7:51-52 (1992)). Additional methods relevant to the production of multispecific antibody molecules are disclosed in, e.g., Fanger et al., Immunol. Methods 4:72-81 (1994).

Exemplary bispecific antibody and antibody-like molecules comprise (i) two antibodies, one with a specificity to the ligand of the orphan ligand, and another to a second target, that are conjugated together, (ii) a single antibody that has one chain specific to the ligand of the orphan ligand, and a second chain specific to a second molecule, and (iii) a single chain antibody that has specificity to the ligand of the orphan ligand and a second molecule. Typically, the second target/second molecule is a molecule other than the ligand of the orphan ligand.

In certain aspects, antibody derivatives are prepared from the identified antibodies or fragments. Such derivatives can be, e.g., antibodies directly derivatized with radioisotopes or other toxic compounds. In such cases, the labeled monospecific antibody can be injected into the patient, where it can then bind to and kill cells expressing the target antigen, with unbound antibody simply clearing the body. Indirect strategies can also be used, such as the “Affinity Enhancement System” (AES) (see, e.g., U.S. Pat. No. 5,256,395; Barbet et al. (1999) Cancer Biother Radiopharm 14: 153-166; the entire disclosures of which are herein incorporated by reference). This particular approach involves the use of a radiolabeled hapten and an antibody that recognizes both the ligand of the orphan ligand and the radioactive hapten. In this case, the antibody is first injected into the patient and allowed to bind to target cells, and then, once unbound antibody is allowed to clear from the blood stream, the radiolabeled hapten is administered. The hapten binds to the antibody-antigen complex on the overproliferating cells, thereby killing them, with the unbound hapten clearing the body.

The toxins or other compounds can be linked to the antibody directly or indirectly, using any of a large number of available methods. For example, an agent can be attached at the hinge region of the reduced antibody component via disulfide bond formation, using cross-linkers such as N-succinyl 3-(2-pyridyldithio)proprionate (SPDP), or via a carbohydrate moiety in the Fc region of the antibody (see, e.g., Yu et al. (1994) Int. J. Cancer 56: 244; Wong, Chemistry of Protein Conjugation and Cross-linking (CRC Press 1991); Upeslacis et al., “Modification of Antibodies by Chemical Methods,” in Monoclonal antibodies: principles and applications, Birch et al. (eds.), pages 187-230 (Wiley-Liss, Inc. 1995); Price, “Production and Characterization of Synthetic Peptide-Derived Antibodies,” in Monoclonal antibodies: Production, engineering and clinical application, Ritter et al. (eds.), pages 60-84 (Cambridge University Press 1995), Cattel et al. (1989) Chemistry today 7: 51-58, Delprino et al. (1993) J. Pharm. Sci 82: 699-704; Arpicco et al. (1997) Bioconjugate Chemistry 8: 3; Reisfeld et al. (1989) Antihody, Immunicon. Radiopharm. 2: 217; the entire disclosures of each of which are herein incorporated by reference).

Any type of moiety with a cytotoxic or cytoinhibitory effect can be used to create an antibody derivative capable of inhibiting or killing specific cells expressing the ligand of, e.g., an orphan receptor, including radioisotopes, toxic proteins, toxic small molecules, such as drugs, toxins, immunomodulators, hormones, hormone antagonists, enzymes, oligonucleotides, enzyme inhibitors, therapeutic radionuclides, angiogenesis inhibitors, chemotherapeutic drugs, vinca alkaloids, anthracyclines, epidophyllotoxins, taxanes, antimetabolites, alkylating agents, antibiotics, COX-2 inhibitors, SN-38, antimitotics, antiangiogenic and apoptotic agents, particularly doxorubicin, methotrexate, taxol, CPT-11, camptothecans, nitrogen mustards, gemcitabine, alkyl sulfonates, nitrosoureas, triazenes, folic acid analogs, pyrimidine analogs, purine analogs, platinum coordination complexes, Pseudomonas exotoxin, ricin, abrin, 5-fluorouridine, ribonuclease (RNase), DNase I, Staphylococcal enterotoxin-A, pokeweed antiviral protein, gelonin, diphtherin toxin, Pseudomonas exotoxin, and Pseudomonas endotoxin and others (see, e.g., Remington's Pharmaceutical Sciences, 19th Ed. (Mack Publishing Co. 1995); Goodman and Gilman's The Pharmacological Basis of Therapeutics (McGraw Hill, 2001); Pastan et al. (1986) Cell 47: 641; Goldenberg (1994) Cancer Journal for Clinicians 44: 43; U.S. Pat. No. 6,077,499; the entire disclosures of which are herein incorporated by reference). It will be appreciated that a toxin can be of animal, plant, fungal, or microbial origin, or can be created de novo by chemical synthesis.

In one aspect, antibody is derivatized with a radioactive isotope, such as 1-131. Any of a number of suitable radioactive isotopes can be used, including, but not limited to, Indium-111, Lutetium-171, Bismuth-212, Bismuth-213, Astatine-211, Copper-62, Copper-64, Copper-67, Yttrium-90, Iodine-125, Iodine-131, Phosphorus-32, Phosphorus-33, Scandium-47, Silver-111, Gallium-67, Praseodymium-142, Samarium-153, Terbium-161, Dysprosium-166, Holmium-166, Rhenium-186, Rhenium-188, Rhenium-189, Lead-212, Radium-223, Actinium-225, Iron-59, Selenium-75, Arsenic-77, Strontium-89, Molybdenum-99, Rhodium-105, Palladium-109, Praseodymium-143, Promethium-149, Erbium-169, Iridium-194, Gold-198, Gold-199, and Lead-211. In general, the radionuclide preferably has a decay energy in the range of 20 to 6,000 keV, preferably in the ranges 60 to 200 keV for an Auger emitter, 100-2,500 keV for a beta emitter, and 4,000-6,000 keV for an alpha emitter. Also preferred are radionuclides that substantially decay with generation of alpha-particles.

In selecting a cytotoxic moiety for inclusion in the present methods, it is desirable to ensure that the moiety will not exert significant in vivo side effects against life-sustaining normal tissues, such as one or more tissues selected from heart, kidney, brain, liver, bone marrow, colon, breast, prostate, thyroid, gall bladder, lung, adrenals, muscle, nerve fibers, pancreas, skin, or other life-sustaining organ or tissue in the human body. The term “significant side effects”, as used herein, refers to an antibody, ligand or antibody conjugate, that, when administered in vivo, will produce only negligible or clinically manageable side effects, such as those normally encountered during chemotherapy.

### Functional Assays

Antibodies, antibody fragments, or small molecules identified by ITACS to bind to a target ligand and interfere with the binding of the ligand with the orphan receptor may be further evaluated in various functional assays. For example, anti-NKp30L mAbs that prevents the binding of soINKp30-FTL-hFc to cells can be tested for their ability to reduce killing by NK cells expressing NKp30. Moreover, killing of target cells bound by anti-NKp30L may be increased in the presence of certain anti-NKp30L mAbs having an isotype that can bind to activating Fc receptors on effector cells. Functional assays can be configured in many other ways to test the functional effects of mAbs identifying in ITACS screens. For example, identification of mAbs that lead to increased killing of tumor cells, and that may be used for tumor immunotherapy, may involve functional testing in killing assays using as targets a cell line that bind the mAb. Identification of mAbs to be used for treatment of autoimmune, inflammatory diseases can involve screening for mAbs that lead to reduce killing of cells bound by the mAb. Standard functional assays, including in vitro and in vivo assays that are known in the art for evaluating an agent for its efficacy in treating a particular condition (e.g., a particular cancer or autoimmune disease) can also be employed in the present context.

### Characterization of Cell Surface-Associated Ligand

Once an antibody or antibody fragment against an target ligand of an orphan ligand has been identified, the antibody or fragment can be used to retrieve and characterize the unknown ligand. For example, a ligand-expressing cell line can be lysed in detergent (e.g., Triton X-100), followed by immunoprecipitation or affinity chromatography with anti-ligand mAbs. Immuno-precipitated proteins can be separated by SDS-PAGE, allowing excision of individual bands that can be analyzed by micro-sequencing using mass-spec technology. Alternatively, anti-ligand mAbs identified by ITACS may be used to expression clone cDNAs encoding the ligand.

### Formulations

The present invention encompasses pharmaceutical formulations comprising agents binding ligands of orphan ligands, including antibodies or other agents identified by ITACS or fusion proteins described herein, which may also comprise one or more pharmaceutically acceptable carriers. Exemplary formulations are described below.

Another object of the present invention is to provide a pharmaceutical formulation comprising an antibody, antibody fragment, antibody derivative, or small molecule which is present in a concentration from 0.1 mg/ml to 100 mg/ml, and wherein said formulation has a pH from 2.0 to 10.0. The formulation may further comprise a buffer system, preservative(s), tonicity agent(s), chelating agent(s), stabilizers and surfactants. In one embodiment of the invention the pharmaceutical formulation is an aqueous formulation, i.e., a formulation comprising water. Such formulation is typically a solution or a suspension. In a further embodiment of the invention the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

As described above, in one aspect, the agent is an antibody or antibody fragment identified by an ITACS procedure, or a fragment or derivative thereof. In this aspect, an exemplary, non-limiting range for a therapeutically or prophylactically effective amount of an antibody, antibody fragment, or antibody derivative is about 0.1-100 mg/kg, such as about 0.1-50 mg/kg, for example about 0.1-20 mg/kg, and more particularly about 1-10 mg/kg (e.g., at about 0.5 mg/kg (such as 0.3 mg/kg), about 1 mg/kg, or about 3 mg/kg). Generally, such an amount is administered once per day or less (e.g., 2-3 times per week, 1 times per week, or 1 time every two weeks).

In another aspect the pharmaceutical formulation is a freeze-dried formulation, whereto the physician or the patient adds solvents and/or diluents prior to use. In another embodiment the pharmaceutical formulation is a dried formulation (e.g. freeze-dried or spray-dried) ready for use without any prior dissolution. In a further aspect the invention relates to a pharmaceutical formulation comprising an aqueous solution wherein the active agent is present in a concentration from 0.1 mg/ml or above, and wherein said formulation has a pH from about 2.0 to about 10.0.

In another aspect, the pH of the formulation is selected from the list consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and 10.0. The buffer may be selected from, e.g., the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative aspect.

The formulation can further comprise a pharmaceutically acceptable preservative. The preservative may, for example, be selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. In further aspects, the preservative is present in a concentration from 0.1 mg/ml to 20 mg/ml; from 0.1 mg/ml to 5 mg/ml; from 5 mg/ml to 10 mg/ml; or from 10 mg/ml to 20 mg/ml. Each one of these specific preservatives constitutes an alternative aspect of the invention. The use of a preservative in pharmaceutical compositions is well-known to the skilled person. For convenience reference is made to Remington: The Science and Practice of Pharmacy, 19^{th} edition, 1995.

The formulation may further comprise an isotonic agent. For example, the isotonic agent can be selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one embodiment the sugar additive is sucrose. Sugar alcohol is defined as a C₄-C₈ hydrocarbon having at least one—OH group and includes, for example, mannitol, sorbitol, inositol, galactitol, dulcitol, xylitol, and arabitol. In one embodiment the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects achieved using the methods of the invention. In one embodiment, the sugar or sugar alcohol concentration is between about 1 mg/ml and about 150 mg/ml. In further embodiments of the invention the isotonic agent is present in a concentration from 1 mg/ml to 50 mg/ml; from 1 mg/ml to 7 mg/ml; from 8 mg/ml to 24 mg/ml; or from 25 mg/ml to 50 mg/ml. Each one of these specific isotonic agents constitutes an alternative embodiment of the invention. The use of an isotonic agent in pharmaceutical compositions is well-known to the skilled person. For convenience reference is made to Remington: The Science and Practice of Pharmacy, 19^{th} edition, 1995.

The formulation may also comprise a chelating agent. The chelating agent may be selected from, e.g., salts of ethylenediaminetetraacetic acid (EDTA), citric acid, and aspartic acid, and mixtures thereof. In particular aspects, the chelating agent is present in a concentration from 0.1 mg/ml to 5 mg/ml; from 0.1 mg/ml to 2 mg/ml; or from 2 mg/ml to 5 mg/ml. Each one of these specific chelating agents constitutes an alternative embodiment of the invention. The use of a chelating agent in pharmaceutical compositions is well-known to the skilled person. For convenience reference is made to Remington: The Science and Practice of Pharmacy, 19^{th} edition, 1995.

The formulation may further comprise a stabilizer. The use of a stabilizer in pharmaceutical compositions is well-known to the skilled person. For convenience reference is made to Remington: The Science and Practice of Pharmacy, 19^{th} edition, 1995. IN one aspect, compositions of the invention are stabilized liquid pharmaceutical compositions whose therapeutically active components include a polypeptide that possibly exhibits aggregate formation during storage in liquid pharmaceutical formulations. By “aggregate formation” is intended a physical interaction between the polypeptide molecules that results in formation of oligomers, which may remain soluble, or large visible aggregates that precipitate from the solution. By “during storage” is intended a liquid pharmaceutical composition or formulation once prepared, is not immediately administered to a subject. Rather, following preparation, it is packaged for storage, either in a liquid form, in a frozen state, or in a dried form for later reconstitution into a liquid form or other form suitable for administration to a subject. By “dried form” is intended the liquid pharmaceutical composition or formulation is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53). Aggregate formation by a polypeptide during storage of a liquid pharmaceutical composition can adversely affect biological activity of that polypeptide, resulting in loss of therapeutic efficacy of the pharmaceutical composition. Furthermore, aggregate formation may cause other problems such as blockage of tubing, membranes, or pumps when the polypeptide-containing pharmaceutical composition is administered using an infusion system.

The pharmaceutical compositions of the invention may further comprise an amount of an amino acid base sufficient to decrease aggregate formation by the polypeptide during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one embodiment, amino acids to use in preparing the compositions of the invention are those carrying a charged side chain, such as arginine, lysine, aspartic acid, and glutamic acid. Any stereoisomer (i.e., L, D, or a mixture thereof) of a particular amino acid (e.g. methionine, histidine, imidazole, arginine, lysine, isoleucine, aspartic acid, tryptophan, threonine and mixtures thereof) or combinations of these stereoisomers, may be present in the pharmaceutical compositions of the invention so long as the particular amino acid is present either in its free base form or its salt form. In one embodiment the L-stereoisomer is used. Compositions of the invention may also be formulated with analogues of these amino acids. By “amino acid analogue” is intended a derivative of the naturally occurring amino acid that brings about the desired effect of decreasing aggregate formation by the polypeptide during storage of the liquid pharmaceutical compositions of the invention. Suitable arginine analogues include, for example, aminoguanidine, ornithine and N-monoethyl L-arginine, suitable methionine analogues include ethionine and buthionine and suitable cysteine analogues include S-methyl-L cysteine. As with the other amino acids, the amino acid analogues are incorporated into the compositions in either their free base form or their salt form. In a further embodiment of the invention the amino acids or amino acid analogues are used in a concentration, which is sufficient to prevent or delay aggregation of the protein.

In a further aspect, methionine (or other sulphuric amino acids or amino acid analogous) may be added to inhibit oxidation of methionine residues to methionine sulfoxide when the polypeptide acting as the therapeutic agent is a polypeptide comprising at least one methionine residue susceptible to such oxidation. By “inhibit” is intended minimal accumulation of methionine oxidized species over time. Inhibiting methionine oxidation results in greater retention of the polypeptide in its proper molecular form. Any stereoisomer of methionine (L or D) or combinations thereof can be used. The amount to be added should be an amount sufficient to inhibit oxidation of the methionine residues such that the amount of methionine sulfoxide is acceptable to regulatory agencies. Typically, this means that the composition contains no more than about 10% to about 30% methionine sulfoxide. Generally, this can be achieved by adding methionine such that the ratio of methionine added to methionine residues ranges from about 1:1 to about 1000:1, such as 10:1 to about 100:1.

In a further aspect, the invention the formulation further comprises a stabilizer selected from the group of high molecular weight polymers or low molecular compounds. In a further embodiment of the invention the stabilizer is selected from polyethylene glycol (e.g. PEG 3350), polyvinyl alcohol (PVA), polyvinylpyrrolidone, carboxy-/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). Each one of these specific stabilizers constitutes an alternative embodiment of the invention.

The pharmaceutical compositions may also comprise additional stabilizing agents, which further enhance stability of a therapeutically active polypeptide therein. Stabilizing agents of particular interest to the present invention include, but are not limited to, methionine and EDTA, which protect the polypeptide against methionine oxidation, and a nonionic surfactant, which protects the polypeptide against aggregation associated with freeze-thawing or mechanical shearing.

The formulation may further comprise a surfactant. In a further embodiment of the invention the surfactant is selected from a detergent, ethoxylated castor oil, polyglycolyzed glycerides, acetylated monoglycerides, sorbitan fatty acid esters, polyoxypropylene-polyoxyethylene block polymers (eg. poloxamers such as Pluronic® F68, poloxamer 188 and 407, Triton X-100), polyoxyethylene sorbitan fatty acid esters, polyoxyethylene and polyethylene derivatives such as alkylated and alkoxylated derivatives (tweens, e.g. Tween-20, Tween-40, Tween-80 and Brij-35), monoglycerides or ethoxylated derivatives thereof, diglycerides or polyoxyethylene derivatives thereof, alcohols, glycerol, lectins and phospholipids (eg. phosphatidyl serine, phosphatidyl choline, phosphatidyl ethanolamine, phosphatidyl inositol, diphosphatidyl glycerol and sphingomyelin), derivates of phospholipids (eg. dipalmitoyl phosphatidic acid) and lysophospholipids (eg. palmitoyl lysophosphatidyl-L-serine and 1-acyl-sn-glycero-3-phosphate esters of ethanolamine, choline, serine or threonine) and alkyl, alkoxyl (alkyl ester), alkoxy (alkyl ether)-derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (eg. cephalins), glyceroglycolipids (eg. galactopyransoide), sphingoglycolipids (eg. ceramides, gangliosides), dodecylphosphocholine, hen egg lysolecithin, fusidic acid derivatives- (e.g. sodium tauro-dihydrofusidate etc.), long-chain fatty acids and salts thereof C6-C12 (eg. oleic acid and caprylic acid), acylcarnitines and derivatives, N′-acylated derivatives of lysine, arginine or histidine, or side-chain acylated derivatives of lysine or arginine, N′-acylated derivatives of dipeptides comprising any combination of lysine, arginine or histidine and a neutral or acidic amino acid, N′-acylated derivative of a tripeptide comprising any combination of a neutral amino acid and two charged amino acids, DSS (docusate sodium, CAS registry no [577-11-7]), docusate calcium, CAS registry no [128-49-4]), docusate potassium, CAS registry no [7491-09-0]), SDS (sodium dodecyl sulphate or sodium lauryl sulphate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-1-propanesulfonate, anionic (alkyl-aryl-sulphonates) monovalent surfactants, zwitterionic surfactants (e.g. N-alkyl-N,N-dimethylammonio-1-propanesulfonates, 3-cholamido-1-propyldimethylammonio-1-propanesulfonate, cationic surfactants (quaternary ammonium bases) (e.g. cetyl-trimethylammonium bromide, cetylpyridinium chloride), non-ionic surfactants (eg. Dodecyl β-D-glucopyranoside), poloxamines (eg. Tetronic's), which are tetrafunctional block copolymers derived from sequential addition of propylene oxide and ethylene oxide to ethylenediamine, or the surfactant may be selected from the group of imidazoline derivatives, or mixtures thereof. Each one of these specific surfactants constitutes an alternative embodiment of the invention. The use of a surfactant in pharmaceutical compositions is well-known to the skilled person. For convenience reference is made to Remington: The Science and Practice of Pharmacy, 19^{th} edition, 1995.

The formulation may further comprise protease inhibitors such as EDTA (ethylenediamine tetraacetic acid) and benzamidineHCl, but other commercially available protease inhibitors may also be used. The use of a protease inhibitor is particular useful in pharmaceutical compositions comprising zymogens of proteases in order to inhibit autocatalysis.

Other ingredients may also be present in the peptide pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Pharmaceutical compositions containing one or more compounds according to the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

Administration of pharmaceutical compositions according to the invention may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Compositions of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

Compositions of the invention may further be compounded in, or attached to, for example through covalent, hydrophobic and electrostatic interactions, a drug carrier, drug delivery system and advanced drug delivery system in order to further enhance stability of the compound, increase bioavailability, increase solubility, decrease adverse effects, achieve chronotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block co-polymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions there of, well known to those skilled in the art of phase behaviour in lipid-water systems, polymeric micelles, multiple emulsions, self-emulsifying, self-microemulsifying, cyclodextrins and derivatives thereof, and dendrimers.

Compositions of the current invention are useful in the formulation of solids, semisolids, powder and solutions for pulmonary administration of compounds according to the invention, using, for example a metered dose inhaler, dry powder inhaler and a nebulizer, all being devices well known to those skilled in the art.

Compositions of the current invention are specifically useful in the formulation of controlled, sustained, protracting, retarded, and slow release drug delivery systems. More specifically, but not limited to, compositions are useful in formulation of parenteral controlled release and sustained release systems (both systems leading to a many-fold reduction in number of administrations), well known to those skilled in the art. Even more preferably, are controlled release and sustained release systems administered subcutaneous. Without limiting the scope of the invention, examples of useful controlled release system and compositions are hydrogels, oleaginous gels, liquid crystals, polymeric micelles, microspheres, nanoparticles,

Methods to produce controlled release systems useful for compositions of the current invention include, but are not limited to, crystallization, condensation, co-crystallization, precipitation, co-precipitation, emulsification, dispersion, high pressure homogenisation, encapsulation, spray drying, microencapsulating, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Formulation and Delivery (MacNally, E. J., ed. Marcel Dekker, New York, 2000).

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension for the administration of the compound in the form of a nasal or pulmonal spray. As a still further option, the pharmaceutical compositions containing the compound of the invention can also be adapted to transdermal administration, e.g. by needle-free injection or from a patch, optionally an iontophoretic patch, or transmucosal, e.g. buccal, administration.

The compound can be administered via the pulmonary route in a vehicle, as a solution, suspension or dry powder using any of known types of devices suitable for pulmonary drug delivery. Examples of these comprise of, but are not limited to, the three general types of aerosol-generating for pulmonary drug delivery, and may include jet or ultrasonic nebulizers, metered-dose inhalers, or dry powder inhalers (Cf. Yu J, Chien Y W. Pulmonary drug delivery: Physiologic and mechanistic aspects. Crit Rev Ther Drug Carr Sys 14(4) (1997) 395-453).

Based on standardised testing methodology, the aerodynamic diameter (da) of a particle is defined as the geometric equivalent diameter of a reference standard spherical particle of unit density (1 g/cm³). In the simplest case, for spherical particles, da is related to a reference diameter (d) as a function of the square root of the density ratio as described by:

$${d}_{a} = \sqrt{\frac{ρ}{{ρ}_{a}}} d$$

Modifications to this relationship occur for non-spherical particles (cf. Edwards D A, Ben-Jebria A, Langer R. Recent advances in pulmonary drug delivery using large, porous inhaled particles. J Appl Physiol 84(2) (1998) 379-385). The terms “MMAD” and “MMEAD” are well-described and known to the art (cf. Edwards D A, Ben-Jebria A, Langer R and represents a measure of the median value of an aerodynamic particle size distribution. Recent advances in pulmonary drug delivery using large, porous inhaled particles. J Appl Physiol 84(2) (1998) 379-385). Mass median aerodynamic diameter (MMAD) and mass median effective aerodynamic diameter (MMEAD) are used inter-changeably, are statistical parameters, and empirically describe the size of aerosol particles in relation to their potential to deposit in the lungs, independent of actual shape, size, or density (cf. Edwards D A, Ben-Jebria A, Langer R. Recent advances in pulmonary drug delivery using large, porous inhaled particles. J Appl Physiol 84(2) (1998) 379-385). MMAD is normally calculated from the measurement made with impactors, an instrument that measures the particle inertial behaviour in air.

In a further embodiment, the formulation could be aerosolized by any known aerosolisation technology, such as nebulisation, to achieve a MMAD of aerosol particles less than 10 μm, more preferably between 1-5 μm, and most preferably between 1-3 μm. The preferred particle size is based on the most effective size for delivery of drug to the deep lung, where protein is optimally absorbed (cf. Edwards D A, Ben-Jebria A, Langer A, Recent advances in pulmonary drug delivery using large, porous inhaled particles. J Appl Physiol 84(2) (1998) 379-385). Deep lung deposition of the pulmonal formulations comprising the compound may optional be further optimized by using modifications of the inhalation techniques, for example, but not limited to: slow inhalation flow (e.g., 30 L/min), breath holding and timing of actuation.

The term “stabilized formulation” refers to a formulation with increased physical stability, increased chemical stability or increased physical and chemical stability.

The term “physical stability” of the protein formulation as used herein refers to the tendency of the protein to form biologically inactive and/or insoluble aggregates of the protein as a result of exposure of the protein to thermo-mechanical stresses and/or interaction with interfaces and surfaces that are destabilizing, such as hydrophobic surfaces and interfaces. Physical stability of the aqueous protein formulations is evaluated by means of visual inspection and/or turbidity measurements after exposing the formulation filled in suitable containers (e.g. cartridges or vials) to mechanical/physical stress (e.g. agitation) at different temperatures for various time periods. Visual inspection of the formulations is performed in a sharp focused light with a dark background. The turbidity of the formulation is characterized by a visual score ranking the degree of turbidity for instance on a scale from 0 to 3 (a formulation showing no turbidity corresponds to a visual score 0, and a formulation showing visual turbidity in daylight corresponds to visual score 3). A formulation is classified physical unstable with respect to protein aggregation, when it shows visual turbidity in daylight. Alternatively, the turbidity of the formulation can be evaluated by simple turbidity measurements well-known to the skilled person. Physical stability of the aqueous protein formulations can also be evaluated by using a spectroscopic agent or probe of the conformational status of the protein. The probe is preferably a small molecule that preferentially binds to a non-native conformer of the protein. One example of a small molecular spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths.

Other small molecules can be used as probes of the changes in protein structure from native to non-native states. For instance the “hydrophobic patch” probes that bind preferentially to exposed hydrophobic patches of a protein. The hydrophobic patches are generally buried within the tertiary structure of a protein in its native state, but become exposed as a protein begins to unfold or denature. Examples of these small molecular, spectroscopic probes are aromatic, hydrophobic dyes, such as antrhacene, acridine, phenanthroline or the like. Other spectroscopic probes are metal-amino acid complexes, such as cobalt metal complexes of hydrophobic amino acids, such as phenylalanine, leucine, isoleucine, methionine, and valine, or the like.

The term “chemical stability” of the protein formulation as used herein refers to chemical covalent changes in the protein structure leading to formation of chemical degradation products with potential less biological potency and/or potential increased immunogenic properties compared to the native protein structure. Various chemical degradation products can be formed depending on the type and nature of the native protein and the environment to which the protein is exposed. Elimination of chemical degradation can most probably not be completely avoided and increasing amounts of chemical degradation products is often seen during storage and use of the protein formulation as well-known by the person skilled in the art. Most proteins are prone to deamidation, a process in which the side chain amide group in glutaminyl or asparaginyl residues is hydrolysed to form a free carboxylic acid. Other degradations pathways involves formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C., Plenum Press, New York 1992). Oxidation (of for instance methionine residues) can be mentioned as another variant of chemical degradation. The chemical stability of the protein formulation can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC).

Hence, as outlined above, a “stabilized formulation” refers to a formulation with increased physical stability, increased chemical stability or increased physical and chemical stability. In general, a formulation must be stable during use and storage (in compliance with recommended use and storage conditions) until the expiration date is reached.

In one embodiment of the invention the pharmaceutical formulation comprising the compound is stable for more than 6 weeks of usage and for more than 3 years of storage. In another embodiment of the invention the pharmaceutical formulation comprising the compound is stable for more than 4 weeks of usage and for more than 3 years of storage. In a further embodiment of the invention the pharmaceutical formulation comprising the compound is stable for more than 4 weeks of usage and for more than two years of storage. In an even further embodiment of the invention the pharmaceutical formulation comprising the compound is stable for more than 2 weeks of usage and for more than two years of storage.

### Therapeutic Applications

Compositions for use in treating a disorder such as a cancer or an autoimmune disease according to the present invention comprise an agent which binds a ligand to an orphan ligand. Such an agent can be an agent identified by ITACS, or a fusion protein designed according to the present invention.

Compositions according to the invention may also comprise an agent binding to the ligand of an orphan ligand in combination with a second agent effective in treating cancer or autoimmune disease. In embodiments comprising administration of such combinations, the dosage of the agent binding to the ligand may on its own comprise an effective amount and additional agent(s) may further augment the therapeutic benefit to the patient. Alternatively, the combination of the agent binding to the ligand and the second agent may together comprise an effective amount for preventing or treating the syndrome. It will also be understood that effective amounts may be defined in the context of particular treatment regimens, including, e.g., timing and number of administrations, modes of administrations, formulations, etc.

Thus, an agent identified by ITACS, as well as any of the fusion proteins provided by the invention, can also be combined with a large number of anti-cancer therapeutic and/or prophylactic agents and therapies. Non-limiting examples of such agents include fluoropyrimidiner carbamates, such as capecitabine; non-polyglutamatable thymidylate synthase inhibitors; nucleoside analogs, such as tocladesine; antifolates such as pemetrexed disodium; taxanes and taxane analogs; topoisomerase inhibitors; polyamine analogs; mTOR inhibitors (e.g., rapamcyin ester); alkylating agents (e.g., oxaliplatin); lectin inhibitors; vitamin D analogs (such as seocalcitol); carbohydrate processing inhibitors; antimetabolism folate antagonists; thumidylate synthase inhibitors; other antimetabolites (e.g., raltitrexed); ribonuclease reductase inhibitors; dioxolate nucleoside analogs; thimylate syntase inhibitors; gonadotropin-releasing hormone (GRNH) peptides; human chorionic gonadotropin; chemically modified tetracyclines (e.g., CMT-3; COL-3); cytosine deaminase; thymopentin; DTIC; carmustine; carboplatin; vinblastine; temozolomide; vindesine; thymosin-α; histone deacetylase inhibitors (e.g., phenylbutyrate); DNA repair agents (e.g., DNA repair enzymes and related compositions such as Dimericine™ (T4 endonuclease V-containing liposome)); gastrin peptides (and related compositions such as Gastrimmune™); GMK and related compounds/compositions (see, e.g., Knutson, Curr Opin Investig Drugs. 2002 January; 3(1):159-64 and Chapman et al., Clin Cancer Res. 2000 December; 6(12):4658-62); beta-catenin blockers/inhibitors and/or agents that lower the amount of beta-catenin production in preneoplastic or neoplastic cell nuclei (see, e.g., U.S. Pat. No. 6,677,116), agents that upregulate E-cadherin expression (or E-cadherin); agents that reduce slug (beta-catenin-associated) gene expression; agents that block, inhibit, or antagonize PAI-1 or that otherwise modulate urokinase plasminogen activator (uPA) interaction with the uPA receptor; survivins; DNA demethylating agents; “cross-linking” agents such as platinum-related anti-cancer agents (cisplatin, carboplatin, etc.); agents that block antiapoptotic signaling, such as agents that inhibit MAPK and Ras signaling pathways or components thereof (e.g., agents that interfere with the production and/or function of cyclin D); growth suppressive agents, such as an antimetabolite such as Cepecitabine/Xeloda, cytarabine/Ara-C, Cladribine/Leustatin, Fludaraine/Fludara, fluorouracil/5-FU, gemcitabine/Gemzar, mercaptopurine/6-MP, methotrexate/MTX, thioguanine/6-TG, Allopurinol/Zyloprim, etc.; an acylating agent such as Busulfan, Cyclophosphamide, mechlorethamaine, Melphalan, thiotepa, semustine, carboplatin, cisplatin, procarbazine, dacarbazine, Althretamine, Lomustine, Carmustine, Chlorambucil, etc.; a topoisomerase inhibitor such as Camptothecins as Topotecan, Irinotecan; such as Podophyllotoxins as Etoposide/VP16, Teniposide/VP26, etc.; an inhibitor of microtuble and/or spindle formation, such as Vincristine, Vinblastine, Vinorelbine, or Taxane such as Paxlitaxel, Docetaxel, combrestatin, Epothilone B, etc; RRR-alpha-tocopheryl succinate; anthracyclins as Daunorubicin/Cerubidine and Doxorubicin; idarubicin; mitomycins; plicamycin; retinoic acid analogues such as all trans retinoic acid, 13-cis retinoic acid, etc.; inhibitors of receptor tyrosine kinases; inhibitors of ErbB-1/EGFR such as iressa, Erbitux, etc.; inhibitors of ErbB-2/Her2 such as Herceptin, etc.; inhibitors of c-kit such as Gleevec; inhibitors of VEGF receptors such as ZD6474, SU6668, etc.; Inhibitors of ErbB3, ErbB4, IGF-IR, insulin receptor, PDGFRa, PDGFRbeta, Flk2, Flt4, FGFR1, FGFR2, FGFR3, FGFR4, TRKA, TRKC, c-met, Ron, Sea, Tie, Tie2, Eph, Ret, Ros, Alk, LTK, PTK7, etc.; cancer related enzyme inhibitors such as metalloproteinase inhibitors such as marimastat, Neovastat, etc.; cathepsin B; modulators of cathepsin D dehydrogenase activity; glutathione-S-transferases and related compounds such as glutacylcysteine synthetase and lactate dehydrogenase; proteasome inhibitors (e.g., Bortezomib); tyrosine kinase inhibitors; farnesyl transferase inhibitors; HSP90 inhibitors (e.g., 17-allyl amino geld-anamycin) and other heat shock protein-inhibitors; mycophenolate mofetil; mycophenolic acid; asparaginase; calcineurin-inhibitors; TOR-inhibitors; multikine molecules; enkephalins (see, e.g., U.S. Pat. No. 6,737,397); SUI 1248 (Pfizer); BAY 43-9006 (Bayer and Onyx); inhibitors of “lymphocyte homing” mechanisms such as FTY720; Tarceva; Iressa; Glivec; thalidomide; and adhesion molecule inhibitors (e.g., anti-LFA, etc.). Additional anti-neoplastic agents that can be used in the combination composition and combination administration methods of the invention include those described in, e.g., U.S. Pat. Nos. 6,660,309, 6,664,377, 6,677,328, 6,680,342, 6,683,059, and 6,680,306, as well as International Patent Application WO 2003070921.

Where appropriate, one or more of such agents also or alternatively can be conjugated to an identified agent or a fusion protein. Such conjugates are another feature of the invention.

Combination compositions and combination delivery methods also or alternatively can include anti-anergic agents (e.g., small molecule compounds, proteins, glycoproteins, or antibodies that break tolerance to tumor and cancer antigens).

In a particular aspect, the invention provides a combination composition that includes at least one agent identified by ITACS or a fusion protein as described herein and at least one secondary anti-cancer monoclonal antibody. A number of suitable anti-cancer mAbs are known in the art and similar suitable antibodies can be developed against cancer-associated targets. Particular examples of suitable second anti-cancer mAbs include anti-CD20 mAbs (such as Rituximab and HuMax-CD20), anti-Her2 mAbs (e.g., Trastuzumab), anti-CD52 mAbs (e.g., Alemtuzumab and Capath® 1H), anti-EGFR mAbs (e.g., Cetuximab, HuMax-EGFr, and ABX-EGF), Zamyl, Pertuzumab, anti-A33 antibodies (see U.S. Pat. No. 6,652,853), anti-oncofetal protein mAbs (see U.S. Pat. No. 5,688,505), anti-PSMA mAbs (see, e.g., U.S. Pat. No. 6,649,163 and Milowsky et al., J Clin Oncol. 2004 Jul. 1; 22(13):2522-31. Epub 2004 Jun. 1), anti-TAG-72 antibodies (see U.S. Pat. No. 6,207,815), anti-aminophospholipid antibodies (see U.S. Pat. No. 6,406,693), anti-neurotrophin antibodies (U.S. Pat. No. 6,548,062), anti-C3b(i) antibodies (see U.S. Pat. No. 6,572,856), anti-cytokeratin (CK) mAbs, anti-MN antibodies (see, e.g., U.S. Pat. No. 6,051,226), anti-mtsl mAbs (see, e.g., U.S. Pat. No. 6,638,504), anti-PSA antibodies (see, e.g., Donn et al., Andrologia. 1990; 22 Suppl 1:44-55; Sinha et al., Anat Rec. 1996 August; 245(4):652-61; and Katzenwadel et al., Anticancer Res. 2000 May-June; 20(3A):1551-5); antibodies against CA125; antibodies against integrins like integrin beta1; antibodies/inhibitors of VCAM; anti-alpha-v/beta-3 integrin mAbs; anti-kininostatin mAbs; anti-aspartyl (asparaginyl) beta-hydroxylase (HAAH) intrabodies (see, e.g., U.S. Pat. No. 6,783,758); anti-CD3 mAbs (see, e.g., U.S. Pat. Nos. 6,706,265 and 6,750,325) and anti-CD3 bispecific antibodies (e.g., anti-CD3/Ep-CAM, anti-CD3/her2, and anti-CD3/EGP-2 antibodies—see, e.g., Kroesen et al., Cancer Immunol Immunother. 1997 November-December; 45(3-4):203-6); and anti-VEGF mAbs (e.g., bevacizumab). Other possibly suitable second mAb molecules include alemtuzumab, edrecolomab, tositumomab, ibritumomab tiuxetan, and gemtuzumab ozogamicin. In one aspect, the invention provides combination compositions and combination therapies that comprise one or more antibodies, typically monoclonal antibodies, targeted against angiogenic factors and/or their receptors, such as VEGF, bFGF, and angiopoietin-1; and monoclonal antibodies against other relevant targets (see also, generally, Reisfeld et al., Int Arch Allergy Immunol. 2004 March; 133(3):295-304; Mousa et al., Curr Pharm Des. 2004; 10(1):1-9; Shibuya, Nippon Yakurigaku Zasshi. 2003 December; 122(6):498-503; Zhang et al., Mol Biotechnol. 2003 October; 25(2):185-200; Kiselev et al., Biochemistry (Mosc). 2003 May; 68(5):497-513; Shepherd, Lung Cancer. 2003 August; 41 Suppl 1:S63-72; O'Reilly, Methods Mol Biol. 2003; 223:599-634; Zhu et al., Curr Cancer Drug Targets. 2002 June; 2(2):135-56; and International Patent Application WO 2004/035537).

In a further aspect, the invention provides combination compositions and methods that include one or more inhibitors of angiogenesis, neovascularization, and/or other vascularization (such agents are referred to by terms such as anti-angiogenesis agents, anti-angiogenic drugs, etc. herein). Nonlimiting examples of such agents include (individually or in combination) endostatin and angiostatin (reviewed in Marx (2003) Science 301, 452-454) and derivatives/analogues thereof; anti-angiogenic heparin derivatives and related molecules (e.g., heperinase III); VEGF-R kinase inhibitors and other anti-angiogenic tyrosine kinase inhibitors (e.g., SU011248—see Rosen et al., Clinical Oncology; May 31-Jun. 3, 2003, Chicago, Ill., USA (abstract 765)); temozolomide; Neovastat™ (Gingras et al., Invest New Drugs. 2004 January; 22(1):17-26); Angiozyme™ (Weng et al., Curr Oncol Rep. 2001 March; 3(2):141-6); NK4 (Matsumoto et al., Cancer Sci. 2003 April; 94(4):321-7); macrophage migration inhibitory factor (MIF); cyclooxygenase-2 inhibitors; resveratrol (see, e.g., Sala et al., Drugs Exp Clin Res. 2003; 29(5-6):263-9); PTK787/ZK 222584 (see, e.g., Klem, Clin Colorectal Cancer. 2003 November; 3(3):147-9 and Zips et al., Anticancer Res. 2003 September-October; 23(5A):3869-76); anti-angiogenic soy isoflavones (e.g., Genistein—see, e.g., Sarkar and L1, Cancer Invest. 2003; 21 (5):744-57); Oltipraz; thalidomide and thalidomide analogs (e.g., CC-5013—see, e.g., Tohnya et al., Clin Prostate Cancer. 2004 March; 2(4):241-3); other endothelial cell inhibitors (e.g., Squalamine and 2-methoxyestradiol); fumagillin and analogs thereof; somatostatin analogues; pentosan polysulfate; tecogalan sodium; molecules that block matrix breakdown (such as suramin and analogs thereof (see, e.g., Marchetti et al., Int J Cancer. 2003 Mar. 20; 104(2):167-74, Meyers et al., J Surg Res. 2000 Jun. 15; 91(2):130-4, Kruger and Figg, Clin Cancer Res. 2001 July; 7(7):1867-72, and Gradishar et al., Oncology. 2000 May; 58(4):324-33)); dalteparin (Scheinowitz et al., Cardiovasc Drugs Ther. 2002 July; 16(4):303-9); matrix metalloproteinase inhibitors (such as BMS-275291—see Rundhaug, Clin Cancer Res. 2003 February; 9(2):551-4; see generally, Coussens et al. Science 2002; 295:2387-2392); angiocol; anti-PDGF mAbs and other PDGF (platelet derived growth factor) inhibitors; and PEDFs (pigment epithelium derived growth factors).

In another aspect, the invention provides combination compositions and combination administration methods with a hormonal regulating agent, such as an anti-androgen and/or anti-estrogen therapy agent or regimen (see, e.g., Trachtenberg, Can J Urol. 1997 June; 4(2 Supp 1):61-64; Ho, J Cell Biochem. 2004 Feb. 15; 91 (3):491-503), tamoxifen, a progestin, a luteinizing hormone-releasing hormone (or an analog thereof or other LHRH agonist), or an aromatase inhibitor (see, e.g., Dreicer et al., Cancer Invest. 1992; 10(1):27-41). Steroids (often dexamethasone) can inhibit tumour growth or the associated edema (brain tumors) and also can be suitable for combination. One or more agents can be similar provided or combined with an antiandrogene such as Flutaminde/Eulexin; a progestin, such as hydroxyprogesterone caproate, Medroxyprogesterone/Provera, Megestrol acepate/Megace, etc.; an adrenocorticosteroid such as hydrocortisone, prednisone, etc.; a luteinising hormone-releasing hormone (LHRH) analogue such as buserelin, goserelin, etc.; and/or a hormone inhibitor such as octreotide/Sandostatin, etc. In a particular aspect, an agent is combined with an anti-cancer agent that is an estrogen receptor modulator (ERM) such as tamoxifen, idoxifene, fulvestrant, droloxifene, toremifene, raloxifene, diethylstilbestrol, ethinyl estradiol/Estinyl, etc., or a combination of any thereof. Combination compositions and combination administration methods also or alternatively can comprise tamoxifen. Further teachings relevant to cancer immunotherapy are provided in, e.g., Berczi et al., “Combination Immunotherapy of Cancer” in NEUROIMMUNE BIOLOGY, Volume 1: New foundation of Biology, Berczi I, Gorczynski R, Editors, Elsevier, 2001; pp. 417-432.

The present invention also encompasses combined administration of one or more additional agents in concert with an agent binding to a ligand of an orphan ligand for treatment of an autoimmune disease. Such additional agents include agents normally utilized for the particular therapeutic purpose for which the antibody or other agent is being administered, e.g. for treatment of an autoimmune disease. Various cytokines may be employed in such combined approaches. Examples of cytokines include IL-1alpha IL-1beta, IL-2, IL-3, IL-4, IL-5, IL-6, IL-7, IL-8, IL-9, IL-10, IL-11, IL-12, IL-13, IL-15, IL-21, TGF-beta, GM-CSF, M-CSF, G-CSF, TNF-alpha, TNF-beta, LAF, TCGF, BCGF, TRF, BAF, BDG, MP, LIF, OSM, TMF, PDGF, IFN-alpha, IFN-beta, IFN-gamma, or compounds that inhibit any of these cytokines. Cytokines or their inhibitors are administered according to standard regimens, consistent with clinical indications such as the condition of the patient and the relative toxicity of the cytokine.

Any cancer or pre-cancerous condition where tumor cells or transformed cells express a hitherto unidentified cell surface-associated ligand can be suitable for treatment according to the present invention. For example, an antibody targeting the unidentified ligand could be used to elicit a host immune response against, or deliver a cytotoxic drug to, the tumor cells. The cancer or pre-cancerous condition may be any neoplastic disorder, including, but not limited to, such cellular disorders as sarcoma, carcinoma, melanoma, leukemia, and lymphoma, which may include cancers or pre-cancerous conditions in the breast, head and neck, ovaries, bladder, lung, pharynx, larynx, esophagus, stomach, small intestines, liver, pancreas, colon, female reproductive tract, male reproductive tract, prostate, kidneys and central nervous system. The types of antibodies contemplated for cancer therapy include, for example antibodies of the IgG1 isotype in the case of an mAb for treatment of cancer where the mAb is intended to bind to tumor cells and induce their death. Such antibodies could, for example, promote the launch of a host immune attack against the tumor cells or transformed cells via ADCC or CDC. For example, in the case of antibodies against NKp30L, NKp44L, or NKp46L, the preferred mAb would be of the IgG1 isotype, in order to cause elimination of tumor targets expressing NKp30L, NKp44L, or NKp46L.

Any viral infection where infected cells express a hitherto unidentified cell surface-associated ligand can be suitable for treatment according to the present invention. For example, an antibody targeting the unidentified ligand could be used to elicit a host immune response against, or deliver a cytotoxic drug to, the infected cells. Such viral infectious organisms include, but are not limited to, hepatitis type A, hepatitis type B. hepatitis type C, influenza, varicella, adenovirus, herpes simplex type I (HSV-1), herpes simplex type 2 (HSV-2), rinderpest, rhinovirus, echovirus, rotavirus, respiratory syncytial virus, papilloma virus, papilloma virus, cytomegalovirus, echinovirus, arbovirus, huntavirus, coxsackie virus, mumps virus, measles virus, rubella virus, polio virus and human immunodeficiency virus type I or type 2 (HIV-1, HIV-2).

Some activating NK receptors, such as NKG2D, have been implicated as propagators of autoimmune diseases. Their ligands may be expressed at high levels in inflamed tissues, thereby causing stimulation of NK cells. Antibodies that bind such ligands, thereby blocking the binding of such activating receptors, may reduce signs and symptoms of inflammation. In such cases, a blocking, non-depleting mAb may be preferred, such as an IgG4 or IgG2. Any autoimmune disease, i.e., a disease or condition that involves the production of a host immune response to host tissue, where a hitherto unidentified cell surface-associated ligand is involved in the disease mechanism, can be suitable for treatment according to the present invention. For example, an antibody blocking the binding of an NK-cell activating receptor to an target ligand expressed on host cells could reduce or prevent NK cell-mediated killing of the host cells. Antibodies for treatment of inflammatory diseases can be of the IgG4 or IgG2 isotype (in cases where the goal is a blocking mAb that would not cause elimination of cells bearing the target antigen) or an IgG1 (in cases where the goal is to eliminate the antigen-bearing cells). Exemplary autoimmune diseases include, but are not limited to, alopecia greata, ankylosing spondylitis, antiphospholipid syndrome, autoimmune Addison's disease, autoimmune hemolytic anemia, autoimmune hepatitis, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatricial pemphigoid, CREST syndrome, cold agglutinin disease, Crohn's disease, discoid lupus, essential mixed cryoglobulinemia, fibromyalgia-fibromyositis, Graves' disease, Guillain-Barre, Hashimoto's thyroiditis, idiopathic pulmonary fibrosis, idiopathic thrombocytopenia purpura (ITP), IgA nephropathy, insulin-dependent diabetes, juvenile arthritis, lichen planus, Meniere's disease, mixed connective tissue disease, multiple sclerosis, myasthenia gravis, pemphigus vulgaris, pernicious anemia, polyarteritis nodosa, polychondritis, polyglandular syndromes, polymyalgia rheumatica, polymyositis, dermatomyositis, primary gammaglobulinemia, primary biliary cirrhosis, psoriasis, Raynaud's phenomenon, Reiter's syndrome, rheumatic fever, rheumatoid arthritis (RA), sarcoidosis, scleroderma, Sjogren's syndrome, stiff-man syndrome, systemic lupus erythematosus (SLE), Takayasu arteritis, temporal arteritis/giant cell arteritis, ulcerative colitis, uveitis, vasculitis, vitiligo, and Wegener's granulomatosis.

Further aspects and advantages of this invention will be disclosed in the following experimental section, which should be regarded as illustrative and not limiting the scope of this application.

### EXAMPLES

### Example 1

### Preparation of soINKp30-FTL-Fc Construct

This examples describes the preparation of a fusion protein comprising residues 20 to 149 of the full NKp30 sequence (SEQ ID NO:1). The NKp30 portion of the construct includes residues 20-138 of SEQ ID NO:1, corresponding to an extracellular fragment of receptor; residues 139-149 from the neighboring transmembrane region, providing a flexible transmembrane (FTL) region, as well as an alanine (A) linker between the NKp30 fragment and the Fc domain. The alanine is introduced via the cloning strategy. However, glycine or another small “in-offensive” amino acid can work just as well as spacer.

Briefly, total RNA template for cDNA synthesis was purified from peripheral blood mononuclear cells (PBMC) from a healthy donor, using RNAeasy Mini Kit (Qiagen#74104) and DNasel (Sigma#AMP-D1) on-column digestion. NKp30 cDNA, encoding the mature form of NKp30 (i.e. lacking the leader-sequence) was generated by reverse transcription and polymerase chain reaction (PCR)-amplification, using NKp30-specific primers that contained artificial restriction sites for either BglII or NheI. For this, OneStep RT-PCR kit (Qiagen#210210)) was used, essentially according to the manufacturer's protocol. The prepared NKp30 cDNA was treated with BglII and NheI, and ligated into a described mammalian expression vector cut with BamHI and NheI, in frame between sequences encoding the CD5 leader-sequence and the genomic sequence for the Fc portion of human IgG1. The ligation product was amplified in Top10/P3 Chemically Competent Cells (Invitrogen #C5050-03) with ampicilin as a selection marker. The nucleotide sequence of the plasmid insert was verified by termination cycling sequencing (MWG, Ebersberg, Germany).

SoINKp30-Fc protein was produced in COS-7 cells that were cultured serum-free after transient transfection of the plasmid DNA encoding soINKp30-Fc. On day 4 post-transfection, soINKp30-Fc was purified from the tissue-culture medium by affinity chromatography, using protein A agarose-columns. SoINKp30-Fc was eluted from the column with 50 mM Na-Citrate, and subsequently dialysed against PBS. The purity of hFc-protein was assessed by both western blot and coomassie-staining after SDS-PAGE. Amino acid sequence integrity was assessed with MALDI-MS, MALDI-MS/MS, and specific binding to an anti-hNKp30 mAb (clone45 from R&D Systems (cat no #MAB1849)) in ELISA. SoINKp30-Fc was conjugated to Allophycocyanin (APC) with Phycolink APC Conjugation Kit (Prozyme# PJ25K) according to the manufacturer's protocol using desalting column (provided with the kit) for DTT removal and 2 times dialysis against PBS for end purification.

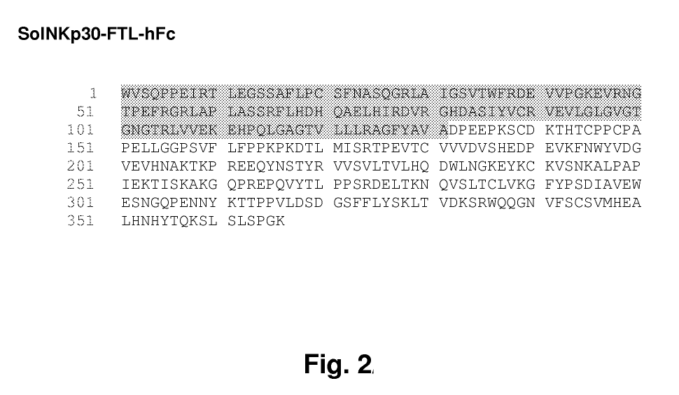

The amino acid sequence of the final soINKp30-FTL-hFc protein is shown in FIG. 2 (SEQ ID NO:4). By similar methods, soluble variants of NKp30 were made, in which the human IgG1 Fc was replaced by mouse IgG1 Fc (SEQ ID NO:5); a leucine residue was added at the N-terminal of soINKp30-FTL-mFc (SEQ ID NO:6); no FTL sequence was included in a soINKp30-mFc fusion protein (SEQ ID NO:7); or where a leucine residue was added to a soINKp30-mFc fusion protein containing no FTL sequence (SEQ ID NO:8). These proteins were produced in the same manner described above.

### Example 2

### Identification of Cells Expressing NKp30L Using soINKp30

This example describes a cell-binding experiment using soINKp30-FTL-hFc to identify cells expressing NKp30L.

Briefly, NKp30L-expressing cell-lines were identified by flow-cytometry (FACS) by their capacity to bind soINKp30-FTL-hFc. Various tumor cell lines were incubated with fixed amounts of fluorescently labeled soINKp30-FTL-hFc (e.g. in the range of 10⁻²-10² μg/ml APC-soINKp30-FTL-hFc), in tissue-culture medium containing 2% FCS, for 30 minutes on ice. Cells were washed and the binding of soINKp30-FTL-hFc to cells was analyzed by flow-cytometry. Alternatively, tumor-cells were incubated with non-fluorescently labeled soINKp30-FTL-hFc, washed, and incubated with fluorescently-labeled secondary antibodies specific for human IgG Fc, washed, and analyzed by flow-cytometry. In both assays, soINKp30-FTL-hFc binding to cells was determined by analyzing the mean-fluorescence bound to individual cells, in comparison with the binding of either secondary antibodies alone, or an irrelevant fluorescently labeled Fc-fusion protein (which does not bind the tumor cells in question). To confirm the specificity of soINKp30-FTL-hFc binding to NKp30L, similar assays were performed with soINKp30-FTL-hFc that was pre-incubated with a molar excess of antibodies known to inhibit NKp30 by binding to NKp30, including anti-NKp30 mAb cat no 210845 from R&D systems. Cells that were able to bind soINKp30-hFc, which could be competed with antibodies known to inhibit NKp30, were designated NKp30L-expressing cells. Cell-lines thus identified as NKp30L-positive included the human erythroleukemia cell line called K562. K562 is an erythroleukemia cell line sensitive to NK-mediated killing, originally derived from a patient with Chronic Myeloid Leukemia.

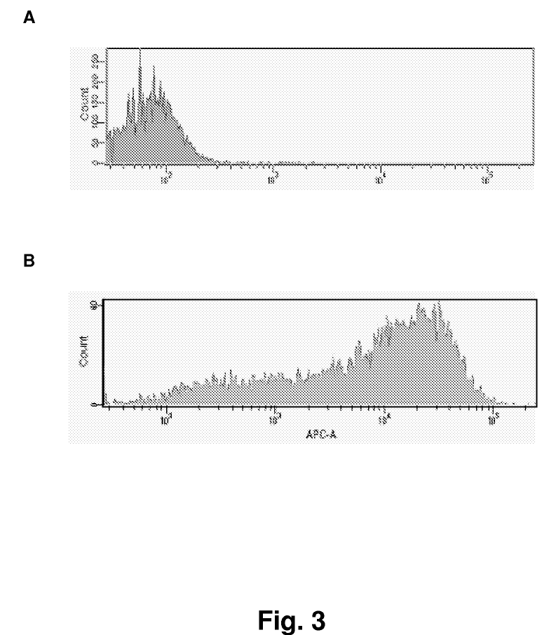

As shown in FIG. 3, soINKp30-FTL-hFc exhibited specific binding to K562 cells. The soINKp30-FTL-Fc protein also bound to additional cell lines, including Daudi, HEK293a, THP-1, CHO-K1, HeLa and COS-7.

### Example 3

### Comparative Cell-Binding of Different soINKp30-Fc Constructs

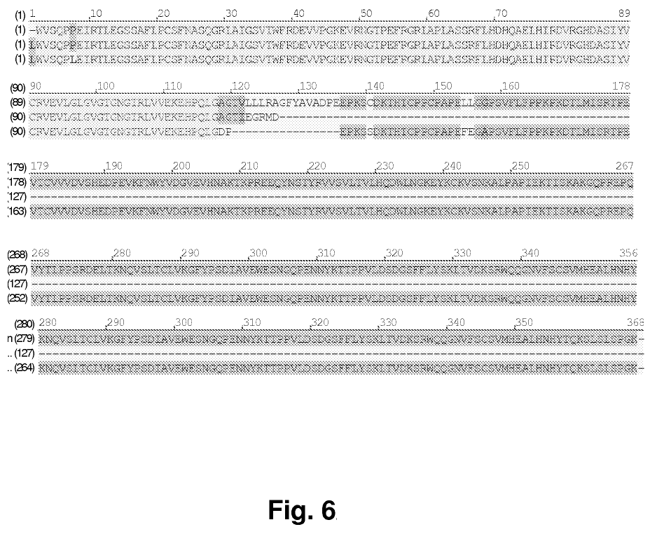

This example describes an experiment designed to compare the cell-binding capabilities of soINKp30-FTL-Fc and the commercially available 1849-NK construct. As shown in FIG. 6, the 1849-NK construct has an N-terminal leucine which is absent from soINKp30-FTL-Fc, and has a different sequence between the extracellular part of NKp30 and human IgG1 Fc; in this region 1849-NK lacks an FTL but instead contains a different, shorter linker sequence.

Briefly, soINKp30-FTL-Fc or 1849-NK proteins (20 ug/ml) were incubated with K562 cells for 45 min on ice. The cells were washed, and incubated with a 1:50 dilution of APC-conjugated Fab′2 donkey anti-human Fc (Jackson Immunoresearch Cat#: 709-136-149) for 30 min on ice. After washing the cells were analyzed by flow cytometry.

As shown in FIG. 4, soINKp30-FTL-Fc had improved binding characteristics over 1849-NK, since it resulted in much stronger fluorescence (x-axis in FIG. 4A) than did 1849-NK (x-axis in FIG. 4B), reflecting an improved strength of binding by soINKp30-FTL-Fc compared to 1849-NK.

### Example 4

### Competition of soINKp30-FTL-Fc with Anti-NKp30 mAb

This example describes a cell-binding competition experiment between soINKp30-FTL-Fc and an anti-NKp30 mAb.

Briefly, soINKp30-Fc was incubated with the anti-NKp30 antibody cl45 (R&D Systems) in RPMI1640, 2% FCS for 30 min before addition to cell suspension. APC conjugated donkey anti-human Fc Fab₂ fragments (Jacksons #709-136-149), used for indirect immunostaining of bound soINKp30-Fc, were added after 45 min and after one wash in Dulbecco's Phosphate Buffered Saline (D-PBS). After 15 min incubation and 3 times wash in Dulbecco's Phosphate Buffered Saline (D-PBS), the cell fluorescence intensities were measured on a FACS CANTO (BD).

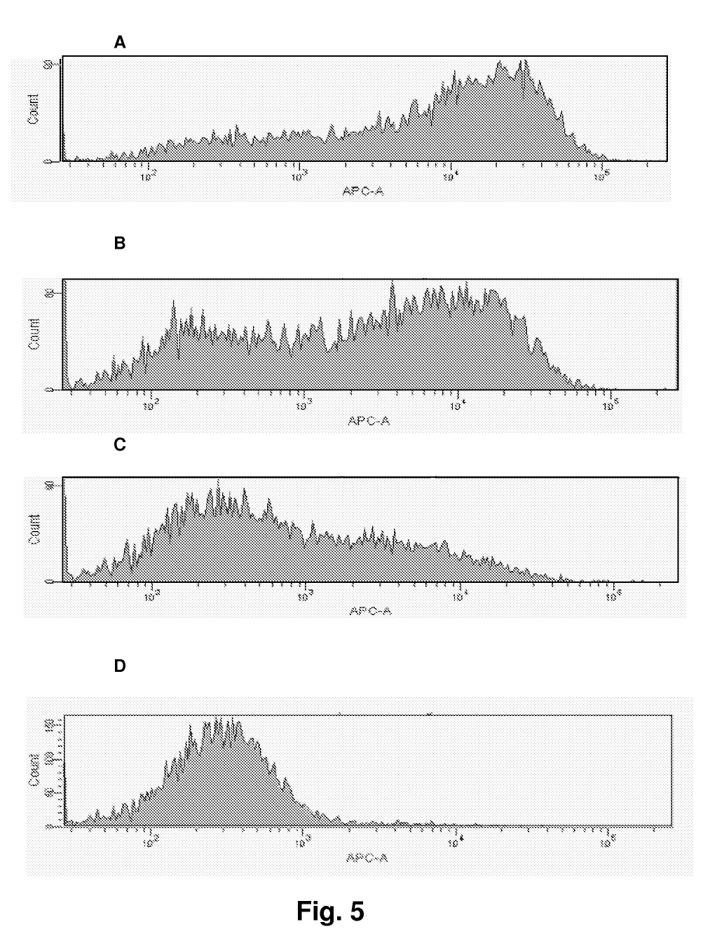

As shown in FIG. 5, the binding of soINKp30-FTL-Fc construct was reduced by increasing amounts of anti-NKp30 (cl45) mAb (90, 180, and 450 μg/ml, FIGS. 5C, 5D, and 5E, respectively).

### Example 5

### Generation of NKp30-mFc(c)

A more traditionally designed NKp30-1 g fusion protein, designated soINKp30-mFc(c) was also produced in order to compare binding to soINKp30-FTL-mFc. The soINKp30-mFc(c) construct was designed to have an N-terminus starting with LWV (Leu-Trp-Val-), and to not contain the FTL (SEQ ID NO:8). The protein was made in the following manner:

The insert sequence was amplified by polymerase chain reaction (PCR). In short, 5 ng of purified NKp30-hFc(A) plasmid template was mixed with 0.6 μM forward primer 5′CACTGCAGCTAGCACTCTGGGTGTCCCAGCCCCCTGAGATTC 3′ (SEQ ID NO:16)

(DNA Technology), 0,6 μM reverse primer

(SEQ ID NO:17)

5′CCAGCAAGATCTGCATCCATCGGCCTTCGATTGTACCAGCCCCTAGCT
GAGG 3′

(DNA Technology) and Taq DNA polymerase (Bioline #BIO-21040) as well as Taq DNA polymerase buffer according to manufacturers protocol. PCR thermocycling conditions consisted of 30 cycles, in which 15 s were given for denaturation at 96° C., 30 s for annealing at 50° C. and 30 s for DNA strand extension at 72° C. The expected amplicon of 405 bp was isolated by agarose gel electrophoresis and digested with SpeI (NEB #R0133S) and BglII (NEB #R0144S) restriction endonucleases. The digested fragment of 370 bp was isolated by agarose gel electrophoresis, and ligated into the same plasmid used for expression of soINKp30-FTL-mFc. Purified plasmid was expressed in COS-7 cells as described for soINKp30-FTL-hFc and -mFc.

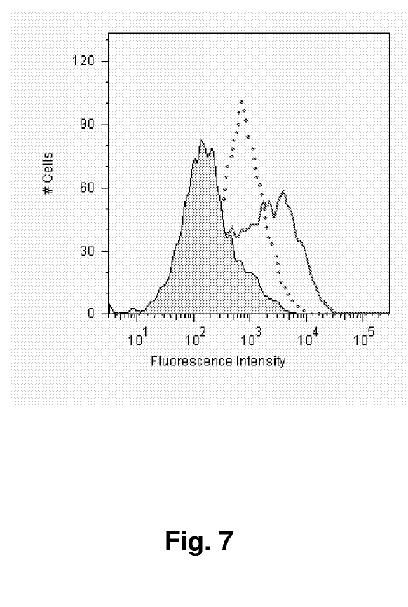

The binding to K562 cells of the two NKp30-Fc fusion proteins was compared by flow cytometry (FIG. 7), and revealed that soINKp30-FTL-mFc bound significantly better than the soINKp30-mFc(c) construct. The amino acid sequence of other variants of NKp30-Fc, designated soINKp30-hFc(ALW), and soINKp30-mFc(ALW), are described in SEQ ID NOS: 9 and 10, respectively. In these construct, the N-terminus of the mature protein starts with the amino acids ALW. Their binding to K562 cells was compared with that of soINKp30-FTL-mFc and soINKp30-mFc(c) by flow cytometry, and were found to bind with similar strength as soINKp30-mFc(c), and much less strongly than soINKp30-FTL-mFc.

### Example 6

### ITACS-Based Identification of Mabs Specific for NKp30L by ITACS

Immunization and Generation of Hybridomas

For the generation of antibodies against the NKp30 ligand, mice were immunized with NKp30L-positive cells (identified as described in Example 2, or with membrane preparations from such cells). Mainly, K562 cell were used, but some initial experiments included also HEK293 and LCL 721.221. The RBF strain of mice were immunized intraperitonally with 2×10⁶ cells or 20 μg membrane extract bi-weekly. Immunizations with membrane extracts were performed with Freund's Complete Adjuvant, whereas whole cells were injected in PBS alone. Commonly, mice were immunized three times in total, and mice were eye-bled ten days after the final immunization to analyze the serum for antibodies against NKp30L-positive cells.

Mice selected for generation of monoclonal antibodies were boosted i.v. with 10 μg membrane extract in PBS, whereas mice immunized with cells were usually not boosted prior to mAb production. Three days after boosting, the spleen was harvested and used for hybridoma production. Spleen cells were fused to FOX-NY myeloma cells (Taggart and Samloff, Science 1983; 219:1228-30) by standard PEG (Harlow and Lane: Using Antibodies, A Laboratory Manual, Cold Spring Harbor Laboratory Press 1999) or electro fusion techniques. The generated hybridoma cells were seeded into 96 well tissue culture plates and the supernatants screened for the presence of antibodies against NKp30L, as described below. Selected clones are subjected to further rounds of subcloning and screening to establish stable hybridoma cell lines.

ITACS Screen

Antibodies that bound NKp30L were identified by flow-cytometry (using a FACSarray, Beckton Dickinson) or Fmat (Applied Biosystems). In either case, the screening assay was a competition assay in which antibodies are screened for their capacity to prevent the binding of soINKp30-FTL-hFc to NKp30L-expressing tumor cell-lines (e.g. K562). For this, tissue-culture supernatants from hybridomas (produced as described above) were incubated with fixed amounts of NKp30L-expressing tumor-cells (e.g 10⁴ K562 cells), for 30 minutes on ice, in 96-well plates. Subsequently, a fixed amount of fluorescently labeled soINKp30-hFc was added to each well (0.1 μg/ml APC-conjugated soINKp30-FTL-hFc), which was then incubated for another 30 minutes on ice. After incubation, cells were washed to remove unbound proteins, and analyzed by flow-cytometry or Fmat. In both assays, soINKp30-FTL-hFc binding to cells was determined by analyzing the mean fluorescence of individual cells. Antibodies were considered to be NKp30L-binding antibodies when they reduced or prevented soINKp30-FTL-hFc-binding to tumor-cells in comparison with the binding of soINKp30-hFc to tumor-cells which had not been pre-incubated with hybridoma supernatants. FIG. 8 shows representative results of such a screen, leading to identification of two anti-NKp30L mAbs. The nature and identity of NKp30L can then be determined by characterizing the antigen(s) recognized by these antibodies.

### EXEMPLARY FEATURES

1. Method of identifying an antibody that binds to a cell surface-associated target ligand of an orphan ligand that is an orphan NK cell receptor, which method comprises:
(a) immunizing at least one vertebrate animal with a first preparation of target cells to which the orphan ligand binds;
(b) preparing at least one test antibody from an antibody-producing cell from the spleen of the vertebrate animal; and
(c) selecting any test antibody that competes with the orphan ligand in binding to a second preparation of target cells as an antibody that binds to a cell surface-associated target ligand of the orphan ligand.
   2. The method of clause 1, wherein the selecting comprises
comparing the binding of a test antibody to the second preparation of target cells in the presence and absence of a reference agent comprising a soluble portion of the orphan ligand, and
identifying any test antibody where the binding is lower in the presence of the reference agent than in the absence of the reference agent.
   3. The method of clause 1, wherein the selecting comprises
comparing the binding of a reference agent comprising a soluble portion of the orphan ligand to the second preparation of target cells in the presence and absence of a test antibody, and
identifying any test antibody where the binding is lower in the presence of the antibody than in the absence of the antibody.
   4. The method of any of clauses 2 or 3, wherein the reference agent is a full-length orphan receptor, an extracellular fragment of the orphan ligand, or a fusion or hybrid protein comprising a soluble portion of the orphan ligand.
   5. The method of clause 4, wherein the fusion or hybrid protein comprises a soluble portion of the orphan ligand covalently bound to an antibody Fc domain, optionally via a linker.
   6. The method of clause 5, wherein the fusion or hybrid protein further comprises at least one amino acid residue of a transmembrane portion of the orphan ligand.
   7. The method of any of clauses 2-3, wherein the reference agent is a full-length orphan ligand attached to a cell membrane or a solid support.
   8. The method of any of clauses 2-3, wherein the reference agent is a soluble portion of the orphan ligand attached to a solid support.
   9. The method of any of clauses 2-8, wherein at least one of the reference agent and the antibody is labeled with a detectable moiety.
   10. The method of clause 9, wherein the detectable moiety is a fluorescent, luminescent, or radioactive compound.
   11. The method of any of the preceding clauses, wherein the antibody-producing cells are B cells.
   12. The method of any of the preceding clauses, wherein the antibody-producing cells are hybridoma cells.
   13. The method of any of the preceding clauses, wherein each of the first and second preparation of target cells is separately selected from intact cells and cell membranes.
   14. The method of any of the preceding clauses, wherein the first and second preparation of target cells are from the same cell line.
   15. The method of any of the preceding clauses, wherein the vertebrate animal is a mouse or rat.
   16. The method of any of the preceding clauses, wherein the orphan ligand is an NK cell activating receptor.
   17. The method of clause 16, wherein the NK cell activating receptor is NKp30, NKp44, NKp46, NKp80, or CD69.
   18. The method of clause 17, wherein the NK cell activating receptor is NKp30.
   19. The method of any of the preceding clauses, wherein the antibody selected in (c) blocks the binding of the orphan ligand to the cell surface-associated ligand.
   20. Method of identifying an antibody or antibody fragment that blocks the binding of a cell surface-associated target ligand to an orphan ligand, which method comprises identifying an antibody according to the method of any of the preceding clauses, and selecting any antibody that reduces the binding between the cell-surface-associated target ligand to the orphan ligand in a dose-dependent fashion.
   21. Method of producing an antibody that binds to a cell surface-associated target ligand of an orphan ligand, comprising the steps of:
(a) identifying an antibody according to the method of any of clauses 1-20, and
(b) producing the antibody from the antibody producing cells.
   22. Method of producing an antibody that binds to a cell surface-associated target ligand of an orphan ligand, comprising the steps of:
(a) identifying an antibody according to the method of any of clauses 1-20;
(b) preparing a nucleic acid encoding the antibody;
(c) transforming a host cell with the nucleic acid; and
(d) culturing the host cell of clause so that the nucleic acid is expressed and the antibody is produced.
   23. The method of clause 22, further comprising recovering the antibody from the host cell culture.
   24. Method of identifying an antibody that binds to a cell surface-associated target ligand of a second ligand, which method comprises:
(a) immunizing at least one vertebrate animal with a first preparation of target cells to which the second ligand binds;
(b) preparing test antibodies from antibody-producing cells from the spleen of the vertebrate animal; and
(c) selecting any antibody that competes with the second ligand in binding to a second preparation of target cells as an antibody that binds to a cell surface-associated target ligand of the second ligand.
   25. The method of clause 24, wherein the second ligand is CD83.
   26. Method of identifying an antibody or antibody fragment that binds to a cell surface-associated target ligand of an orphan ligand, which method comprises:
(a) providing a preparation of target cells to which the orphan ligand binds;
(b) screening a library of test antibodies or antibody fragments for an antibody competing with the orphan ligand in binding to the target cell preparation; and
(c) selecting an antibody or antibody fragment competing with the orphan ligand.
   27. The method of clause 21, wherein the library is a phage-display library.
   28. Method of identifying an antibody that binds to a cell surface-associated target ligand of an NK cell receptor selected from NKp30, NKp44, and NKp46, which method comprises:
(a) providing a cell line to the NK cell receptor binds;
(b) immunizing at least one vertebrate animal with a preparation of cells or cell membranes of the cell line;
(c) isolating B cells from the spleen of the at least one vertebrate animal;
(d) preparing hybridomas from the isolated B cells:
(e) evaluating the binding of an antibody from each hybridoma to cells of the cell line, in (i) the presence and (ii) the absence of a fusion protein comprising a soluble portion of the NK cell receptor and an antibody Fc domain; and
(f) selecting an antibody where the binding in (i) is lower than the binding in (ii).
   29. Method of identifying an antibody that binds to a cell surface-associated target ligand of an NK cell receptor selected from NKp30, NKp44, and NKp46, which method comprises:
(a) providing a cell line to the NK cell receptor binds;
(b) immunizing at least one vertebrate animal with a preparation of cells or cell membranes of the cell line;
(c) isolating B cells from the spleen of the at least one vertebrate animal;
(d) preparing hybridomas from the isolated B cells:
(e) evaluating the binding of a fusion protein comprising a soluble portion of the NK cell receptor and an antibody Fc domain to cells of the cell line in (i) the presence and (ii) the absence of an antibody from each hybridoma; and
(f) selecting an antibody from a hybridoma where the binding in (i) is lower than the binding in (ii).
   30. The method of any of clauses 28 and 29, wherein the NK cell receptor is NKp30.
   31. The method of clause 30, wherein the fusion protein comprises the sequence of any of SEQ ID NOS:4, 5, and 6.
   32. A method of identifying an agent that binds to NKp30L, which method comprises:
(a) providing a plurality of test agents;
(b) evaluating the binding of each test agent to a cell line expressing NKp30L in (i) the presence and (ii) the absence of a soluble NKp30-Fc fusion protein comprising at least one amino acid residue from the transmembrane region of NKp30; and
(c) selecting a test agent where the binding in (i) is lower than the binding in (ii).
   33. A method of identifying an agent that binds to NKp30L, which method comprises:
(a) providing a plurality of test agents;
(b) evaluating the binding of a soluble NKp30-Fc fusion protein comprising at least one amino acid residue from the transmembrane region of NKp30 to a cell line expressing NKp30L in the presence of each test agent; and
(c) selecting any test agent where the binding is lower in the presence of the test agent than in the absence of any test agent.
   34. An antibody, antibody fragment, or agent identified according to the method of any of the preceding clauses.
   35. A fragment or derivative of the antibody of clause 34.
   36. A fusion protein comprising a soluble ligand-binding fragment of an NK cell receptor selected from NKp30, NKp44, and NKp46, covalently linked to an antibody Fc domain via a linker comprising at least one amino acid residue from the transmembrane region of the NK cell receptor.
   37. The fusion protein of clause 36, wherein the NK cell receptor is NKp30 and the fusion protein comprises at least amino acid residues 20-138 of SEQ ID NO:1.
   38. The fusion protein of any of clauses 36 and 37, wherein the linker comprises at least amino acid residues 140-141 of SEQ ID NO:1.
   39. The fusion protein of any of clauses 36-38, wherein the C-terminal residue of the soluble ligand-binding fragment corresponds to a residue selected from 141, 142, 143, 144, 145, 146, 147, 148, and 149 of SEQ ID NO:1.
   40. The fusion protein of any of clauses 36-39, wherein the C-terminal residue of the soluble ligand-binding fragment corresponds to residue 149 of SEQ ID NO:1.
   41. The fusion protein of any of clauses 36-40, wherein the N-terminal residue of the soluble ligand-binding fragment corresponds to residue 20 in SEQ ID NO:1.
   42. The fusion protein of any of clauses 36-41, wherein the N-terminal residue of the soluble ligand-binding fragment corresponds to residue 20 in SEQ ID NO:1, and the C-terminal residue of the soluble ligand-binding fragment corresponds to residue 149 of SEQ ID NO:1.
   43. The fusion protein of clause 37, comprising any of SEQ ID NOS:4 and 5.
   44. The fusion protein of clause 37, consisting of any of SEQ ID NOS:4 and 5.
   45. The fusion protein of clause 36, wherein the NK cell receptor is NKp44, and the fusion protein comprises at least amino acid residues 193-195 of SEQ ID NO:2.
   46. The fusion protein of clause 45, wherein the C-terminal residue of the soluble ligand-binding fragment corresponds to a residue selected from 195, 196, 197, 198, 199, 200, 201, 202, or 203 of SEQ ID NO:2.
   47. The fusion protein of clause 36, wherein the NK cell receptor is NKp46, and the fusion protein comprises at least amino acid residue 256-258 of SEQ ID NO:3.
   48. The fusion protein of clause 47, wherein the C-terminal residue of the soluble ligand-binding fragment corresponds to a residue selected from 258, 259, 260, 261, 262, 263, 264, 265, and 266 of SEQ ID NO:3.
   49. Method of inhibiting NK cell-mediated killing of a cell, the method comprising contacting the antibody, antibody fragment, antibody derivative, or agent of any of clauses 34-35, or the fusion protein of any of clauses 36-49, with a cell expressing the cell surface-associated ligand.
   50. Method of treating cancer or a viral disease, the method comprising administering to a subject an effective amount of the antibody, antibody fragment, antibody derivative, or agent of any of clauses 34-35, or the fusion protein of any of clauses 36-49, wherein the antibody, antibody fragment, antibody derivative, or fusion protein is conjugated to a cytotoxic moiety or is capable of eliciting and ADCC or CDC response.
   51. The method of clause 50, wherein the cytotoxic moiety is a toxin or a radioactive compound.
   52. Method of treating an autoimmune disease, the method comprising administering to a subject an effective amount of the antibody, antibody fragment, antibody derivative, or agent of any of clauses 34-35, or the fusion protein of any of clauses 36-49.

All references, including publications, patent applications and patents, cited herein are hereby incorporated by reference to the same extent as if each reference was individually and specifically indicated to be incorporated by reference and was set forth in its entirety herein.

All headings and sub-headings are used herein for convenience only and should not be construed as limiting the invention in any way, Any combination of the above-described elements in all possible variations thereof is encompassed by the invention unless otherwise indicated herein or otherwise clearly contradicted by context.

The terms “a” and “an” and “the” and similar referents as used in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

The citation and incorporation of patent documents herein is done for convenience only and does not reflect any view of the validity, patentability and/or enforceability of such patent documents,

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

This invention includes all modifications and equivalents of the subject matter recited in the aspects or claims presented herein to the maximum extent permitted by applicable law.

1. Method of identifying an antibody that binds to a cell surface-associated target ligand of an orphan ligand that is an orphan NK cell receptor, which method comprises:
(a) immunizing at least one vertebrate animal with a first preparation of target cells to which the orphan ligand binds;
(b) preparing at least one test antibody from an antibody-producing cell from the spleen of the vertebrate animal; and
selecting any test antibody that competes with the orphan ligand in binding to a second preparation of target cells as an antibody that binds to a cell surface-associated target ligand of the orphan ligand.

2. The method of claim 1, wherein the selecting comprises
comparing the binding of a test antibody to the second preparation of target cells in the presence and absence of a reference agent comprising a soluble portion of the orphan ligand, and
identifying any test antibody where the binding is lower in the presence of the reference agent than in the absence of the reference agent.

3. The method of claim 1, wherein the selecting comprises
comparing the binding of a reference agent comprising a soluble portion of the orphan ligand to the second preparation of target cells in the presence and absence of a test antibody, and
identifying any test antibody where the binding is lower in the presence of the antibody than in the absence of the antibody.

4. The method of claim 2, wherein the reference agent is a full-length orphan receptor, an extracellular fragment of the orphan ligand, or a fusion or hybrid protein comprising a soluble portion of the orphan ligand.

5. The method of claim 4, wherein the fusion or hybrid protein comprises a soluble portion of the orphan ligand covalently bound to an antibody Fc domain, optionally via a linker.

6. The method of claim 5, wherein the fusion or hybrid protein further comprises at least one amino acid residue of a transmembrane portion of the orphan ligand.

7. The method of claim 2, wherein the reference agent is a full-length orphan ligand attached to a cell membrane or a solid support.

8. The method of claim 2, wherein the reference agent is a soluble portion of the orphan ligand attached to a solid support.

9. The method of claim 2, wherein at least one of the reference agent and the antibody is labeled with a detectable moiety.

10. The method of claim 9, wherein the detectable moiety is a fluorescent, luminescent, or radioactive compound.

11. The method of claim 1, wherein the antibody-producing cells are B cells.

12. The method of claim 1, wherein the antibody-producing cells are hybridoma cells.

13. The method of claim 1, wherein each of the first and second preparation of target cells is separately selected from intact cells and cell membranes.

14. The method of claim 1, wherein the first and second preparation of target cells are from the same cell line.

15. The method of claim 1, wherein the vertebrate animal is a mouse or rat.

16. The method of claim 1, wherein the orphan ligand is an NK cell activating receptor.

17. The method of claim 16, wherein the NK cell activating receptor is NKp30, NKp44, NKp46, NKp80, or CD69.

18. The method of claim 17, wherein the NK cell activating receptor is NKp30.

19. The method of claim 1, wherein the antibody selected in (c) blocks the binding of the orphan ligand to the cell surface-associated ligand.

20. Method of identifying an antibody or antibody fragment that blocks the binding of a cell surface-associated target ligand to an orphan ligand, which method comprises identifying an antibody according to the method of any of the preceding claims, and selecting any antibody that reduces the binding between the cell-surface-associated target ligand to the orphan ligand by at least 20%.

21. Method of producing an antibody that binds to a cell surface-associated target ligand of an orphan ligand, comprising the steps of:
identifying an antibody according to the method of claim 1, and
producing the antibody from the antibody producing cells.

22. Method of producing an antibody that binds to a cell surface-associated target ligand of an orphan ligand, comprising the steps of:
identifying an antibody according to claim 1; preparing a nucleic acid encoding the antibody; transforming a host cell with the nucleic acid; and
culturing the host cell of claim so that the nucleic acid is expressed and the antibody is produced.

23. The method of claim 22, further comprising recovering the antibody from the host cell culture.

24. Method of identifying an antibody that binds to a cell surface-associated target ligand of a second ligand, which method comprises:
immunizing at least one vertebrate animal with a first preparation of target cells to which the second ligand binds;
preparing test antibodies from antibody-producing cells from the spleen of the vertebrate animal; and
selecting any antibody that competes with the second ligand in binding to a second preparation of target cells as an antibody that binds to a cell surface-associated target ligand of the second ligand.

25. The method of claim 24, wherein the second ligand is CD83.

26. Method of identifying an antibody or antibody fragment that binds to a cell surface-associated target ligand of an orphan ligand, which method comprises:
providing a preparation of target cells to which the orphan ligand binds;
screening a library of test antibodies or antibody fragments for an antibody competing with the orphan ligand in binding to the target cell preparation; and
selecting an antibody or antibody fragment competing with the orphan ligand.

27. The method of claim 21, wherein the library is a phage-display library.

28. Method of identifying an antibody that binds to a cell surface-associated target ligand of an NK cell receptor selected from NKp30, NKp44, and NKp46, which method comprises:
providing a cell line to the NK cell receptor binds;
immunizing at least one vertebrate animal with a preparation of cells or cell membranes of the cell line;
isolating B cells from the spleen of the at least one vertebrate animal;
preparing hybridomas from the isolated B cells:
evaluating the binding of an antibody from each hybridoma to cells of the cell line, in (i) the presence and (ii) the absence of a fusion protein comprising a soluble portion of the NK cell receptor and an antibody Fc domain; and
selecting an antibody where the binding in (i) is lower than the binding in (ii).

29. Method of identifying an antibody that binds to a cell surface-associated target ligand of an NK cell receptor selected from NKp30, NKp44, and NKp46, which method comprises:
providing a cell line to the NK cell receptor binds;
immunizing at least one vertebrate animal with a preparation of cells or cell membranes of the cell line;
isolating B cells from the spleen of the at least one vertebrate animal;
preparing hybridomas from the isolated B cells:
evaluating the binding of a fusion protein comprising a soluble portion of the NK cell receptor and an antibody Fc domain to cells of the cell line in (i) the presence and (ii) the absence of an antibody from each hybridoma; and
selecting an antibody from a hybridoma where the binding in (i) is lower than the binding in (ii).

30. The method of claim 28, wherein the NK cell receptor is NKp30.

31. The method of claim 30, wherein the fusion protein comprises the sequence of any of SEQ ID NOS:4, 5, and 6.

32. A method of identifying an agent that binds to NKp30L, which method comprises:
providing a plurality of test agents;
evaluating the binding of each test agent to a cell line expressing NKp30L in (i) the presence and (ii) the absence of a soluble NKp30-Fc fusion protein comprising at least one amino acid residue from the transmembrane region of NKp30; and
selecting a test agent where the binding in (i) is lower than the binding in (ii).

33. A method of identifying an agent that binds to NKp30L, which method comprises:
providing a plurality of test agents;
evaluating the binding of a soluble NKp30-Fc fusion protein comprising at least one amino acid residue from the transmembrane region of NKp30 to a cell line expressing NKp30L in the presence of each test agent; and
selecting any test agent where the binding is lower in the presence of the test agent than in the absence of any test agent.

34. (canceled)

35. (canceled)

36. A fusion protein comprising a soluble ligand-binding fragment of an NK cell receptor selected from NKp30, NKp44, and NKp46, covalently linked to an antibody Fc domain via a linker comprising at least one amino acid residue from the transmembrane region of the NK cell receptor.

37. The fusion protein of claim 36, wherein the NK cell receptor is NKp30 and the fusion protein comprises at least amino acid residues 20-138 of SEQ ID NO:1.

38. The fusion protein of claim 36, wherein the linker comprises at least amino acid residues 140-141 of SEQ ID NO:1.

39. The fusion protein of any of claim 36, wherein the C-terminal residue of the soluble ligand-binding fragment corresponds to a residue selected from 141, 142, 143, 144, 145, 146, 147, 148, and 149 of SEQ ID NO:1.

40. The fusion protein of claim 36, wherein the C-terminal residue of the soluble ligand-binding fragment corresponds to residue 149 of SEQ ID NO:1.

41. The fusion protein of claim 36, wherein the N-terminal residue of the soluble ligand-binding fragment corresponds to residue 20 in SEQ ID NO:1.

42. The fusion protein of claim 36, wherein the N-terminal residue of the soluble ligand-binding fragment corresponds to residue 20 in SEQ ID NO:1, and the C-terminal residue of the soluble ligand-binding fragment corresponds to residue 149 of SEQ ID NO:1.

43. The fusion protein of claim 37, comprising any of SEQ ID NOS:4 and 5.

44. The fusion protein of claim 37, consisting of any of SEQ ID NOS:4 and 5.

45. The fusion protein of claim 36, wherein the NK cell receptor is NKp44, and the fusion protein comprises at least amino acid residues 193-195 of SEQ ID NO:2.

46. The fusion protein of claim 45, wherein the C-terminal residue of the soluble ligand-binding fragment corresponds to a residue selected from 195, 196, 197, 198, 199, 200, 201, 202, or 203 of SEQ ID NO:2.

47. The fusion protein of claim 36, wherein the NK cell receptor is NKp46, and the fusion protein comprises at least amino acid residue 256-258 of SEQ ID NO:3.

48. The fusion protein of claim 47, wherein the C-terminal residue of the soluble ligand-binding fragment corresponds to a residue selected from 258, 259, 260, 261, 262, 263, 264, 265, and 266 of SEQ ID NO:3.

49. Method of inhibiting NK cell-mediated killing of a cell, the method comprising contacting the fusion protein of claim 36, with a cell expressing the cell surface-associated ligand.

50. Method of treating cancer or a viral disease, the method comprising administering to a subject an effective amount of the fusion protein of claim 36, wherein the fusion protein is conjugated to a cytotoxic moiety or is capable of eliciting and ADCC or CDC response.

51. The method of claim 50, wherein the cytotoxic moiety is a toxin or a radioactive compound.

52. Method of treating an autoimmune disease, the method comprising administering to a subject an effective amount of the fusion protein of claim 36.
